(19) United States
(12) Patent Application Publication
(10) Pub. No.: US 2026/0335376 A1
(43) Pub. Date: Oct. 29, 2026

(54) MUCOADHESIVE PROBIOTIC BACKPACKS WITH ROS NANO-SCAVENGERS ENHANCE THE BACTERIOTHERAPY FOR INFLAMMATORY BOWEL DISEASES

(71) Applicant: Wisconsin Alumni Research Foundation, Madison, WI (US)

(72) Inventors: Quanyin Hu, Waunakee, WI (US); Jun Liu, Madison, WI (US)

(73) Assignee: Wisconsin Alumni Research Foundation, Madison, WI (US)

(21) Appl. No.: 19/102,446

(22) PCT Filed: Aug. 9, 2023
(86) PCT No.: PCT/US2023/029844

§ 371 Date: Feb. 10, 2025

Related U.S. Application Data
(60) Provisional application No. 63/396,409, filed on Aug. 9, 2022.

(51) Int. Cl. A61K 35/741; A61K 35/00; A61K 47/69; A61P 1/04

(52) U.S. Cl. CPC A61K 35/741; A61K 47/6939; A61P 1/04; A61K 2035/115

(57) ABSTRACT

A composition of matter for treating inflammatory bowel diseases (IBDs) and a corresponding method for treating IBDs. The composition of matter includes a reactive oxygen species (ROS) scavenger conjugated to a probiotic. The method includes administering the composition of matter to a subject in an anti-IBD-effective amount.

### BACKGROUND

Reactive oxygen species (ROS), which are byproducts of aerobic metabolism, are crucial molecules in physiological processes.^{1,2} However, their over-production will cause oxidative stress, which will amplify inflammatory responses and exacerbate inflammatory disorders. This is especially true in the gastrointestinal (GI) tract, resulting in intestinal mucosal layer damage and pathogen invasion. The subsequently triggered immune responses ultimately lead to the onset of inflammatory bowel diseases (IBDs).³⁻⁵ Excessive ROS-induced oxidative stress in the intestines is thought to be a major factor in the pathogenesis and progression of IBDs. Antioxidants are known to scavenge ROS in the intestines and are helpful for the treatment of IBDs.⁶ However, due to their rapid clearance, nonspecific drug biodistribution after systemic administration, and relatively inefficient ROS-scavenging ability, antioxidants demonstrate inconsistent efficacy for treating inflammatory diseases. Furthermore, undesirable drug distribution profiles of antioxidants in normal tissues can cause a variety of adverse effects.^{7,8}

In addition to excessive ROS in the intestines, IBD is associated with dysbiosis of the gut microbiota in the colonic microenvironment.⁹⁻¹¹ In a healthy individual, microbiota provide the host with short-chain fatty acids and essential vitamins while also protecting them from pathogen colonization and invasion. In contrast, a disordered microbiota induces a chronic inflammatory state, increases toxic production, and disrupts the host's metabolism.¹²⁻¹⁴ In previous studies, it is found that orally administered probiotics can colonize colon tissues and aid in the restoration of the normal gut microbiome to treat GI tract-related diseases.^{15,16} Unfortunately, these probiotics are highly sensitive to the harsh environments of the GI tract which limits their viability and retention time in the intestines, leading to decreased therapeutic efficacy.^{17,18} To address the long-felt and unmet need for an effective treatment of IBDs, disclosed herein is a novel platform that can selectively and sustainably scavenge ROS in inflamed colon tissues while also improving probiotic delivery for gut microbiota homeostasis modulation. The platform helps restore a normal gut microenvironment and addresses the fundamental issues underlying effective IBD therapy.

### SUMMARY

Inflammatory bowel diseases (IBDs) are often associated with elevated levels of reactive oxygen species (ROS) and a highly dysregulated gut microbiota. Disclosed herein is a strategy that targets inflamed colon tissues, eliminates over-expressed ROS, and modulates the microbiota balance, thereby restoring the normal homeostasis in colon tissues and thus improving IBD treatment.

Disclosed herein is a composition of matter for treating IBDs, comprising a ROS scavenger conjugated to a probiotic. The ROS scavenger selectively and sustainably scavenges ROS in inflamed colon tissue. The probiotic helps to modulate the microbiota balance in the gut. The ROS scavenger is conjugated to the probiotic to effectively deliver the ROS scavenger to inflamed colon tissues for normalizing ROS levels and minimizing off-target side effects.

In one version of the conjugate, the ROS scavenger is a hyaluronic acid-poly (propylene sulfide (HA-PPS) polymer that is self-assembled to form a nanoparticle (HPN) based on the combined amphiphilic properties. The HA-PPS polymer is formed by conjugating hyaluronic acid (HA) to poly (propylene sulfide) (PPS) with ethylenediamine as a linker. HA is biocompatible and effective in addressing IBDs by modulating the immune response and serving as a potent anti-inflammatory agent. The HPN exhibits improved hydrophilicity and cytoprotective effects while maintaining the robust ROS-scavenging activity of PPS. Further, the HPN self-degrades once the ROS species are consumed, which opens the door for clinical applications.

To achieve colon tissue-targeting effects, the ROS scavenger is conjugated on the surface of a probiotic. The probiotic is encapsulated by one or more coatings to protect the probiotic and improve the viability of the probiotic in oral delivery and prolong the retention time of the probiotic in the intestines. In one version of the disclosure, the probiotic is Escherichia coli Nissle 1917 (EcN), and the coating is Norepinephrine (NE). Oral probiotic delivery of the probiotic is enhanced via encapsulation with NE, which can be auto-oxidized to form a protective film on the probiotic surface. Further, the catecholamine group of NE imparts a robust mucosa adhesive property on the probiotic, resulting in extended retention time in the intestine.

The ROS scavenger is conjugated onto the probiotic with a ROS-responsive linker to facilitate the release of the ROS scavenger from the probiotic in the inflamed colon tissues with elevated ROS level. In certain versions of the disclosure, the ROS-responsive linker has a terminal amine group.

Thus, disclosed herein is a composition of matter for treating inflammatory bowel diseases (IBDs) comprising a reactive oxygen species (ROS) scavenger conjugated to a probiotic.

Also disclosed herein is a composition of matter wherein the ROS scavenger is a hyaluronic acid-poly (propylene sulfide (HA-PPS) polymer that is self-assembled to form a nanoparticle (HPN). In one version, the HA-PPS polymer may be formed by conjugating hyaluronic acid to poly (propylene sulfide) using ethylenediamine as a linker.

The probiotic may be encapsulated by one or more coatings, such as a coating comprising norepinephrine.

In preferred versions of the composition of matter, the probiotic is Escherichia coli Nissle 1917.

It is preferred, but not required, that the ROS scavenger is conjugated to the probiotic with an ROS-responsive linker. The ROS-responsive linker preferably (but not necessarily) has a terminal amine group. A particularly preferred ROS-responsive linker is 2,2′-(propane-2,2-diylbis(sulfanediyl))bis(ethan-1-amine).

In all versions of the composition of matter, it is preferred (but not required) that at least a portion of the ROS-responsive linker degrades in the presence of ROS to release the ROS scavenger from the probiotic.

Also disclosed herein is a pharmaceutical composition for treating IBDs, comprising the composition of matter disclosed herein.

Also disclosed herein is a method of treating IBDs, comprising administering to a subject a therapeutically effective amount of the composition of matter disclosed herein. The composition of matter may be orally administered to reach the gastrointestinal tract of a subject. A therapeutically effective amount of the composition of matter typically comprises between about 10⁶ and about 10¹² CFU probiotic and between about 1 to about 100 mg/kg ROS scavenger. Dosages above and below these ranges are explicitly included within the disclosure.

The objects and advantages of the disclosure will appear more fully from the following detailed description of the preferred embodiment of the disclosure made in conjunction with the accompanying drawings.

### BRIEF DESCRIPTION OF THE DRAWINGS

FIGS. 1A-IC. Schematic illustration of preparation of HPN-NE-EcN and its mechanism for IBD treatment. (FIG. 1A) Preparation of HPN by self-assembly of HA-PPS molecule, encapsulation of EcN with the NE layer, and conjugation of HPN on the surface of EcN. (FIGS. 1B and 1C) The prepared HPN-NE-EcN exerts ROS-scavenging activity by oxidizing sulfur atoms in PPS to form sulfoxides and then further oxidizing to form sulphones (i). Furthermore, the encapsulated NE layer, which mimics mussel adhesive foot proteins, endows EcN a strong mucoadhesive ability and extends the retention time of EcN in the intestines (ii), allowing for enhanced bacteriotherapy through restoring gut microbiome homeostasis (iii).

FIG. 2. Schematic of the physiochemical transformation of PPS from hydrophobic to hydrophilic under ROS conditions.

FIG. 3. ¹H NMR spectrum of PPS.

FIG. 4. ESI-mass of PPS.

FIGS. 5A-5G. Preparation and characterization of HPN. (FIG. 5A) General procedure for the synthesis of HA-PPS conjugate. (FIG. 5B) Size distribution of HPN measured by DLS (n=3). (FIG. 5C) TEM images of HPN. (FIG. 5D) DCF fluorescence intensity after incubation of DCFHDA (50 μM) with H₂O₂ (1 mM) in the presence of HPN (1 mg/mL), PPS (80 μg/mL), or PBS. (FIG. 5E) Viability of HCT116 cells after 72 h of incubation in H₂O₂ conditions (100 μM) with or without HPN treatment (0.5 mg/mL). (FIGS. 5F and 5G) The normalized proliferation rate of EcN (FIG. 5F) and E. coli K12 (FIG. 5G) after culturing in LB medium containing H₂O₂ (100 μM) with or without HPN treatment (0.5 mg/mL). Statistical analysis was performed using one-way ANOVA. **P<0.01, ***P<0.001.

FIG. 6. ¹H NMR spectrum of HA-PPS.

FIG. 7. ¹H NMR spectra of PPS (red) and PPS after incubation with H₂O₂ overnight (green).

FIGS. 8A-8B. Size (FIG. 8A) and zeta potential (FIG. 8B) of the HPN measured by DLS.

FIG. 9. The levels of hyaluronan breakdown product after incubating HA or HPN with hyaluronidase at different time points measured using a colorimetric method.

FIGS. 10A-10J. Preparation and characterization of NE-EcN and HPN-NE-EcN against environmental assaults. (FIG. 10A) Sizes and (FIG. 10B) zeta potentials of EcN and NE-EcN measured by DLS. Data are presented as the mean±SEM (n=3). (FIG. 10C) TEM images of EcN and NE-EcN. Scale bar, 1 μm. (FIGS. 10D-10F) Survival of EcN (left bar) and NE-EcN (right bar) after exposure to the following conditions: (FIG. 10D) SGF (pH 1.5) supplemented with pepsin (0.32%), (FIG. 10E) bile salt (0.4%), and (FIG. 10F) SIF (pH 6.8) supplemented with trypsin (10 mg/mL). (FIG. 10G) Schematic illustration for HPN-NE-EcN preparation. (FIG. 10H) Representative TEM image of HPN-NE-EcN. Scale bar, 1 μm. (FIG. 10I) LSCM images of HPN-NE-EcN. Scale bar, 10 μm. (FIG. 10J) The growth curves of EcN, NE-EcN, and HPN-NE-EcN in LB medium at 37 °C monitored by OD 600 nm values in 30 min intervals via a microplate reader. Data are presented as the mean±SEM (n=3). Statistical analysis was performed using Student's t-test. ***P<0.001.

FIGS. 11A-11D. The universality of the NE coating strategy on E. coli CC. (FIG. 11A) TEM images of E. coli CC and NE-E. coli CC. Scale bar, 1 μm. (FIGS. 1B-11D) Evaluation of E. coli CC (left bar) and NE-E. coli CC (right bar) against environmental stresses: (FIG. 11B) SGF (pH 1.5) supplemented with pepsin (0.32%), (FIG. 11C) Bile salts (0.4%), (FIG. 11D) SIF (pH 6.8) supplemented with trypsin (10 mg/mL).

FIG. 12. The general procedures for the preparation of the ROS-responsive linker.

FIG. 13. ¹H NMR spectrum of intermediate 2 in the synthesis of the ROS-responsive linker.

FIG. 14. ¹H NMR spectrum of intermediate 3 in the synthesis of the ROS-responsive linker.

FIG. 15. ¹H NMR spectrum of the ROS-responsive linker.

FIG. 16. ¹H NMR spectra of the ROS-responsive linker and the linker after incubation with H₂O₂ for 2 h and 4 h.

FIG. 17. Loading efficacy of HPN on NE-EcN.

FIGS. 18A-18G. Mucoadhesive capability of HPN-NE-EcN. (FIG. 18A) Bioluminescence images of mice administered with various luciferase-expressed bacteria formulations at different time points. (FIG. 18B) Bioluminescence images of mice GI tracts at 48 h post-administration of various luciferase-expressed bacterial formulations. (FIG. 18C) Region-of-Interest analysis of bioluminescence intensities of the mice GI tracts at 48 h. Data are presented as mean±SEM (n=3). (FIG. 18D) Fluorescence images of mice after administration of HPN or HPN-NE-EcN for 4 h and 12 h. (FIG. 18E) Region-of-Interest analysis of fluorescence intensities of the mice being given HPN or HPN-NE-EcN. Data are presented as mean±SEM (n=3). (FIG. 18F) LSCM images of mice colon tissues after 48 h administration of FAM-labeled EcN or HPN-NE-EcN. The blue color represents Hoechst 33342-stained colon tissues, and the green color represents FAM-labeled EcN cells. (FIG. 18G) Fluorescence images of mice colon tissues at 12 h post-administration of Alexa fluor 647-labeled HPN or HPN-NE-EcN. The blue color represents colonic cells stained with Hoechst 33342, and the cyan color represents the Alexa fluor 647-labeled HPN. Statistical analysis was performed using one-way ANOVA, respectively. *P<0.05, ***P<0.001.

FIG. 19. Bioluminescence decay curve of bioluminescence intensities of the mice in FIG. 10A at all pre-determined timepoints through Region-of-Interest analysis.

FIG. 20. The distribution of the HPN in major organs after HPN-NE-EcN treatment for 12 h.

FIGS. 21A-21I. Preventative efficacy of HPN-NE-EcN against DSS-induced mouse colitis model. (FIG. 21A) Schematic of the administration schedule. Various HPN and EcN formulations were administered every two days by oral gavage, and 3% DSS was given in drinking water from day 0 to day 7. (FIG. 21B) Percentage weight changes for the colitis-bearing mice treated with various EcN and HPN formulations. (FIG. 21C) Colon tissue images, and (FIG. 21D) quantified colon length in different groups. (FIG. 21E) Representative histological images of colon tissues stained with H&E, and (FIG. 21F) histopathology scores of colon tissues based on H&E images. (FIG. 21G) Relative colonic MPO activities in colon tissues. (FIG. 21H) LSCM images of DCF fluorescence, and (FIG. 21I) Quantified DCF fluorescence intensities in colon tissues after incubating with DCFHDA to reflect ROS levels in different groups through ImageJ software. Scale bar, 250 μm. Statistical analysis was performed using one-way ANOVA. *P<0.05, **P<0.01.

FIG. 22. ROS levels in colon tissues in the DSS⁻, HPN-NE-EcN, and DSS⁺ groups imaged by LSCM. The blue color represents colonic cells, which were labeled by Hoechst 33342, and the green color indicated the fluorescence from DCF which represents ROS levels. Scale bar, 250 μm.

FIG. 23. Histological images of the heart, liver, spleen, lung and kidney tissues stained with H&E in DSS⁻ and HPN-NE-EcN groups. Scale bar, 100 μm.

FIGS. 24A-24G. Therapeutic efficacy of HPN-NE-EcN against DSS-induced mouse colitis model. (FIG. 24A) Schematic of the treatment plan. The mice were administered 3% DSS from day 0 to day 7 to induce mouse colitis. Afterward, different formulations of EcN and HPN were given to the mice for 4 days to assess their therapeutic efficacy. (FIG. 24B) Percent weight changes of colitis-bearing mice in different groups after therapeutic treatment. (FIG. 24C) Colon images, and (FIG. 24D) quantified colon length in different groups. (FIG. 24E) Representative H&E-stained colon tissue images, and (FIG. 24F) histopathology scores of colon tissues to evaluate colon damage. Scale bar, 100 μm. (FIG. 24G) Relative MPO activities in colon tissues to reflect colonic inflammation levels in different groups. Statistical analysis was performed using one-way ANOVA. *P<0.05, **P<0.01, ***P<0.001.

### DETAILED DESCRIPTION

### Abbreviations and Definitions

EcN=Escherichia coli Nissle 1917. HA=Hyaluronic acid. HPN=HA-PPS nanoparticles. NE=Norepinephrine; PPS=Poly (propylene sulfide). ROS=Reactive oxygen species.

As used herein, the term “inflammatory bowel disease (IBD) refers to a disorder or disease characterized by inflammatory activity in the gastrointestinal tract. Examples of IBDs include, without limitation, Crohn's disease (both distal and proximal), ulcerative colitis, indeterminate colitis, microscopic colitis, collagenous colitis, idiopathic inflammation of the small and/or proximal intestine and IBD-related diarrhea.

As used herein, the term “probiotic” refers to a substantially pure bacteria (i.e., a single isolate, or, e.g., live bacterial cells, conditionally lethal bacterial cells, inactivated bacterial cells, killed bacterial cells, spores, recombinant carrier strains), or a mixture of desired bacteria, bacteria components or bacterial extract, or bacterial-derived products (natural or synthetic bacterial-derived products such as bacterial antigens or metabolic products) and may also include any additional components that can be administered to a mammal.

As used herein, the term “method’ refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “prophylactic,” “prevent,” and “preventing” refer to delaying the onset of or reducing the likelihood of developing a disease or disorder or one or more symptoms thereof, as compared to an untreated control population.

As used herein a “pharmaceutical composition” refers to a preparation of the composition of matter described herein. The purpose of a pharmaceutical composition is to facilitate administration of an active ingredient to a subject.

As used herein, an “therapeutically effective amount” refers to a nontoxic but sufficient amount of a composition of material to provide a desired systemic or local effect. The effective amount will vary with the nature of the composition and constituent parts, the age and physical condition of the end user, the severity of the disease, the duration of the treatment, the nature of concurrent therapy, the particular pharmaceutically acceptable carrier utilized, and like factors.

As used herein, the term “administering” refers to a method for bringing the composition of matter into an area or a site in the GI tract that is affected by the IBD.

As used herein, the term “about” refers to ±10%.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

As used herein, the singular forms “a,” “an,” and “the” include plural referents unless the content clearly dictates otherwise.

As used herein, the term “or” is an inclusive “or” operator and is equivalent to the term “and/or” unless the context clearly dictates otherwise.

The term “based on” is not exclusive and allows for being based on additional factors not described, unless the context clearly dictates otherwise.

The elements and method steps described herein can be used in any combination whether explicitly described or not, unless otherwise specified or clearly implied to the contrary by the context in which the referenced combination is made.

All combinations of method steps as used herein can be performed in any order, unless otherwise specified or clearly implied to the contrary by the context in which the referenced combination is made.

The methods and compositions disclosed herein can comprise, consist of, or consist essentially of the essential elements and limitations of the method described herein, as well as any additional or optional ingredients, components, or limitations described herein or otherwise useful in treating IBDs. The disclosure provided herein may be practiced in the absence of any element or step which is not specifically disclosed herein.

It is understood that the disclosure is not confined to the particular elements and method steps herein illustrated and described, but embraces such modified forms thereof as come within the scope of the claims.

### Composition of Matter

Disclosed herein is a composition of matter for treating IBDs, comprising a ROS scavenger conjugated to a probiotic. The ROS scavenger selectively and sustainably scavenges ROS in inflamed colon tissue. The probiotic helps to modulate the microbiota balance. Thus, the composition of matter assists to restore a normal gut microenvironment and address the fundamental issues for effective IBD therapy.

A ROS scavenger is a moiety capable of acting as a scavenger of, or reacting with, superoxide (O2-) or other reactive oxygen species (ROS) including hydroxyl radicals, peroxynitrite, hypochlorous acid and hydrogen peroxide. In the broadest sense of the disclosure, any ROS scavenger having activity against over-expressed ROS in the gastrointestinal tract is contemplated to be useful.

In one version of the disclosure, the ROS scavenger is a polymer of hyaluronic acid-poly (propylene sulfide) (HA-PPS) that is self-assembled into a nanoparticle (HPN).

Poly (propylene sulfide) (PPS) is a hydrophobic polymer to scavenge ROS.¹⁹ The sulfur atoms of PPS are easily oxidized by ROS to form a sulfoxide which will be further oxidized to produce a sulphone.²⁰ This inherent PPS reactivity with ROS provides PPS antioxidant properties that can serve as a highly efficient tool for scavenging ROS.²¹ Moreover, the PPS polymer contains multiple ROS-reacting sites capable of scavenging multiple ROS molecules compared to small antioxidant molecules. However, the clinical application of PPS is limited because it is highly hydrophobic that makes it difficult for in vivo application.²²

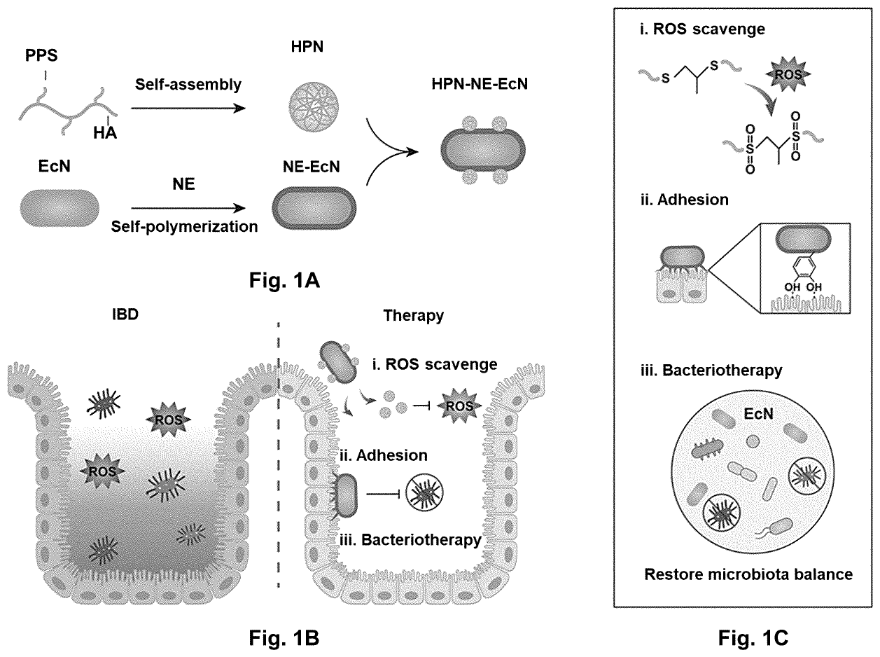

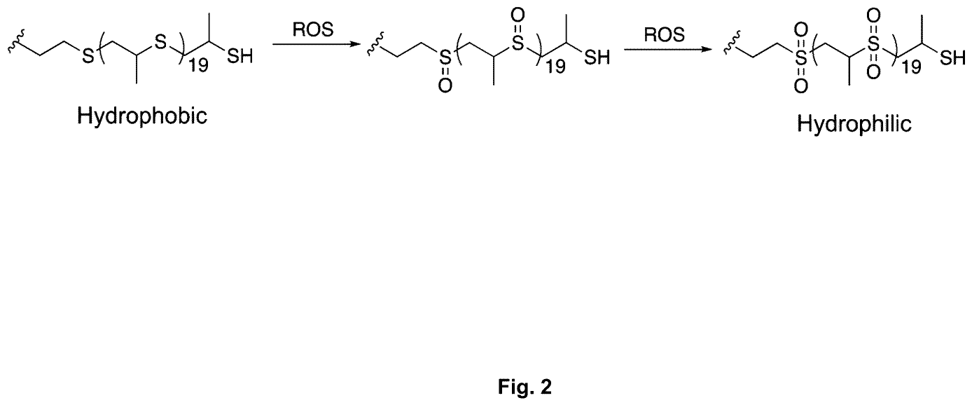

Thus, a ROS scavenger disclosed herein is a hyaluronic acid (HA)-PPS conjugate which is self-assembled to a nanoparticle (HPN) based on the amphiphilic properties that exist when HA and PPS are combined (FIG. 1A). HA is used because it is a biocompatible glycosaminoglycan biopolymer that can improve IBDs by modulating the immune response and serving as a potent anti-inflammatory agent.²³ The HA-PPS polymer is synthesized using ethylenediamine as a linker and the HA-PPS conjugate in aqueous buffer is self-assembled to form HPN. See the Example Section for detailed method of synthesis and characterization of the HPN. The HPN exhibits improved hydrophilicity and cytoprotective effects while maintaining the robust ROS-scavenging activity of PPS. Once the ROS is consumed and the sulfur atoms are oxidized to sulphone, the HPN will self-degrade due to the transformation of PPS from a hydrophobic to hydrophilic state (FIG. 2).²⁴ This transformation enhances the safety of HPN and opens the door for clinical applications.

The second component of the composition of matter according to the disclosure is a probiotic. The digestive systems of humans and other mammals include bacteria essential to the health of the gastrointestinal system and overall-heath of the individual. Beneficial types of bacteria provide various health benefits, including enhancing digestion, nutrient absorption, bowel function, and natural immunity. Also, beneficial bacteria may produce vitamins and, moreover, may inhibit the growth of pathogenic microorganisms, such as pathogenic bacteria, viruses, and/or protozoa. Beneficial bacteria may inhibit the growth of such undesirable microorganisms, for example, by secreting bacteriocins and/or substances that reduce gastrointestinal tract pH, thereby making the gastrointestinal environment less hospitable to pathogenic microorganisms. Disruption of the balance of the normal intestinal flora can lead to conditions ranging from mild gastrointestinal symptoms to serious infection.

Examples of probiotics useful in the present disclosure include, without limitation, bacteria selected from the group consisting of Escherichia, Bifidobacterium, Lactobacillus, Streptococcus, Propionibacterium, Enterococcus, and mixture thereof. Particular non-limiting examples of probiotics include certain strains of Escherichia coli, Arthrobacter agilis, Arthrobacter citreus, Arthrobacter globiformis, Arthrobacter leuteus, Arthrobacter simplex, Azotobacter chroococcum, Azotobacter paspali, Azospirillum brasiliencise, Azospirillium lipoferum, Bacillus brevis, Bacillus macerans, Bacillus pumilus, Bacillus polymyxa, Bacillus subtilis, Bacteroides lipolyticum, Bacteroides succinogenes, Brevibacterium lipolyticum, Brevibacterium stationis, Bacillus laterosporus, Bacillus bifidum, Bacillus laterosporus, Bifidophilus infantis, Streptococcus thermophilous, Bifodophilus longum, Bifidobacteria animalis, Bifidobacteria bifidus, Bifidobacteria breve, Bifidobacteria longum, Kurtha Zopfil, Lactobacillus paracasein, Lactobacillus acidophilus, Lactobacillus planetarium, Lactobacillus salivarius, Lactobacillus rueteri, Lactobacillus bulgaricus, Lactobacillus helveticus, Lactobacillus casei, Lactobacillus rhamnosus, Lactobacillus sporogenes, Lactococcus lactis, Myrothecium vertrucaris, Pseudomonas calcis, Pseudomonas dentrificans, Pseudomonas flourescens, Pseudomonas glathei, Phanerochaete chrysosporium, Saccharomyces boulardii, Streptomyces fradiae, Streptomyces cellulosae, Stretpomyces griseoflavus, and combinations thereof.

Preferably, the probiotic is encapsulated by one or more coatings to protect the probiotic and improve the viability of the probiotic in oral delivery and prolong the retention time of the probiotic in the intestines. Examples of the coatings useful in the disclosure include, without limitation, norepinephrine (NE), polyacrylamides, phthalate derivatives such as acid phthalates of carbohydrates, amylose acetate phthalate, cellulose acetate phthalate (CAP), other cellulose ester phthalates, cellulose ether phthalates, hydroxypropylcellulose phthalate (HPCP), hydroxypropylmethylcellulose phthalate (HPMCP), methylcellulose phthalate, polyvinyl acetate phthalate (PVAP), polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and malice acid copolymers, polyacrylic acid derivatives such as acrylic acid and acrylic ester copolymers, poly methacrylic acid and esters thereof, polyacrylic methacrylic acid copolymers, vinyl acetate and crotonic acid copolymers, methacrylic acid and ethyl acrylate copolymers, methyl methacrylate and methacrylate copolymers, shellac, poly(d,l-lactide-co-glycolide) (PLGA), chitosan, carboxymethyl-ethylcellulose (CMEC), cellulose acetate trimellitiate (CAT), hydroxypropylmethyl cellulose (HPMC), ethyl cellulose, and methyl cellulose.

In one version of the disclosure, the probiotic is Escherichia coli Nissle 1917 (EcN), a probiotic strain that can colonize in colon tissues and re-establish intestinal flora homeostasis in the setting of IBD. To improve the oral probiotic delivery to the colon for enhanced bacteriotherapy, EcN may be encapsulated with norepinephrine (NE), which could be auto-oxidized to form a poly-norepinephrine film on the probiotic surface to protect it from external environmental assaults. The Example Section shows a method of encapsulating EcN with the NE layer and demonstrates the universality of the NE layer coating for other live cells, e.g., E. coli CC. The catecholamine group of NE, which is found rich in mussel adhesive foot proteins and responsible for the strong adhesive properties in mussels,^{25,26} endows probiotics robust mucosa adhesive property and extended retention time of probiotics in the intestines without influencing probiotics' growth and proliferation for enhanced therapeutic efficacy (FIG. 1B).

The ROS scavenger is conjugated onto the probiotic to effectively deliver the ROS scavenger to inflammatory colon tissues for normalizing ROS levels and minimizing off-target side effects. The ROS scavenger may be conjugated onto the probiotic with a ROS-responsive linker, which facilitates the release of the ROS scavenger from the probiotic in the inflamed colon tissues with elevated ROS level. Examples of the ROS-responsive linker useful in the present disclosure include, but not limited to materials described in Liang J. and Liu B., Bioeng Transl Med. 2016, 1(3): 239-251 and Gao F. and Xiong Z., Front. Chem. 2021, Article 649048, including, e.g., polymers containing thioether, selenium/tellurium, thioketal, polysaccharide, aminoacrylate, boronic ester, peroxalate ester and polyproline. In one embodiment, the ROS-responsive linker has a terminal amine group, e.g., 2,2′-(Propane-2,2-diylbis(sulfanediyl))bis(ethan-1-amine).

Overall, the composition of matter disclosed herein not only has the ability to prolong the retention time of probiotics in the intestines for enhanced bacteriotherapy, but it can also specifically deliver and slow-release the ROS scavenger in the intestines for improved ROS-scavenging capabilities (FIG. 1C).

### Pharmaceutical Composition

Disclosed herein are pharmaceutical compositions that include any of the composition of matter comprising a ROS scavenger conjugated onto a probiotic in the present disclosure. In one version, the ROS scavenger is a polymer of hyaluronic acid-poly (propylene sulfide) (HA-PPS) that is self-assembled to a nanoparticle (HPN) and conjugated onto the probiotic with a ROS-responsive linker.

The pharmaceutical composition is preferably formulated and administered as a liquid formulation. The liquid formulation typically comprises a suspension of the ROS scavenger conjugated probiotic in an aqueous solution. The aqueous solution may be a water solution, a buffered solution, and the like.

In some embodiments, the pharmaceutical composition may be dried. Drying may comprise spray drying, fluid bed drying, or freeze-drying. For example, a suspension of the composition of matter is treated with proteins, maltodextrins, trehalose, and optionally, other stabilizing or free-protecting agents like ascorbic acid, to form a viscous paste, which is submitted to freeze-drying. The so-obtained material can be grinded to appropriate size in suitable dosage forms.

The pharmaceutical compositions for use in accordance with the present disclosure may be formulated in conventional manner using one or more pharmaceutically acceptable carriers comprising excipients, diluents, and auxiliaries, which facilitate processing of the active ingredients into preparations which can be used pharmaceutically. Such carriers and auxiliaries enable the pharmaceutical compositions to be formulated as tablets, pills, dragees, capsules, liquids, gels, syrups, slurries, suspensions, and the like, for oral ingestion by a subject.

For oral preparations, the composition of matter can be used alone or in combination with appropriate additives to make tablets, pills, dragees, capsules, liquids, gels, syrups, slurries, suspensions, and the like, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Method of Use

Disclosed herein are methods of treating IBDs, comprising administering to a subject a therapeutically effective amount of the composition of matter or the pharmaceutical composition disclosed herein.

The methods are contemplated to be useful for the treatment, prevention, amelioration, or reduction of symptoms of IBDs. A variety of individuals are treatable according to the subject methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

An effective amount of the composition of matter (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual (e.g., IBDs) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the composition of matter or pharmaceutical composition.

A therapeutically effective amount, according to the method of the present disclosure, preferably ranges between about 10⁶ and about 10¹² CFU probiotic per administration, more preferably between about 10⁷ and about 10¹⁰ CFU probiotic per administration, more preferably between about 10⁷ and about 10⁹ CFU probiotic per administration, and most preferably about 10⁸ CFU probiotic per administration; and between about 1 to about 100 mg/kg ROS scavenger per administration, more preferably between about 10 to about 50 mg/kg ROS scavenger per administration, more preferably between about 20 to about 40 mg/kg ROS scavenger per administration, and most preferably about 30 mg/kg ROS scavenger; wherein the ROS scavenger is conjugated onto the probiotic.

The number of administrations according to the present disclosure preferably ranges between 1 and 10 administrations per day, more preferably between 1 and 5 administrations per day, and most preferably between 2 and 4 administrations per day. The overall amount of probiotic that is administered daily, preferably ranges between 10⁷ and 10⁹ CFU per day; and the overall amount of the ROS scavenger that is administered daily, preferably ranges between 20 to 40 mg/kg per day; wherein the ROS scavenger is conjugated onto the probiotic.

### Examples

### Summary

In this Example, we synthesized a polymer of hyaluronic acid-poly (propylene sulfide) (HA-PPS) and developed ROS-scavenging nanoparticles (HPN) that could effectively scavenge ROS and protect colonic epithelial cells as well as commensal bacteria from ROS-induced damages. To achieve colon tissue targeting effects, the HPN nanoparticles were conjugated on the surface of Escherichia coli Nissle 1917 (EcN), a well-known probiotic strain that can colonize in colon tissues. Moreover, EcN has previously demonstrated the ability to re-establish intestinal flora homeostasis in the setting of IBD. To enhance the bacteriotherapy of EcN, we further encapsulated EcN cells with a poly-norepinephrine (NE) layer that can protect EcN against environmental assaults to improve the viability of EcN in oral delivery and prolong the retention time of EcN in the intestines due to its strong mucoadhesive capability. Notably, this novel platform of HPN-NE-EcN can also resist clearance and extend the retention time of HPN in colon tissues for persistent ROS-scavenging. In the dextran sulfate sodium (DSS) induced mouse colitis models, HPN-NE-EcN showed significantly enhanced prophylactic and therapeutic efficacy for the treatment of colitis.

### Materials and Methods

Materials. All commercially available reagents were used without further purification in this Example. Anhydrous solvents were dried through routine protocols. All chemical reactions were carried out under a nitrogen atmosphere in dry glassware with magnetic stirring. Analytical TLC was carried out employing 0.25 mm silica gel plates (GF254), and column chromatography was carried out on 200-300 mesh silica gel. The NMR spectra were recorded on a Bruker 400 spectrometer. The main chemicals and biological material used in this study are listed below: hydrochloric acid (HCl, 37%, Lab Chem), N-Hydroxysuccinimide (NHS, Alfa Aesar), Sodium hydroxide (NaOH, Alfa Aesar), Trypsin from porcine pancreas (Sigma), Pepsin powder (Fisher science), Bile salts (Sigma), Ampicillin (Sigma), Dextran sulfate sodium salt (DSS, MW 40,000, Alfa Aesar), LB broth (Fisher bioreagents), Agar (Fisher bioreagents), Hydrogen peroxide solution (H₂O₂, Sigma), Sodium chloride (NaCl, Fisher chemical), Hoechst 33342 trihydrochloride (Life Technologies), Cell counting kit-8 (CCK-8, Apexbio), 2′,7′-Dichlorofluorescin diacetate (DCFH-DA, Sigma), Isopropyl β-D-1-thiogalactopyranoside (IPTG, Chem-impex). The Lysogeny-Broth (LB) liquid media was prepared using 25 g of LB broth powder in 1 L of deionized (DI) water and was then autoclaved. LB agar plates were prepared on dishes using 20 mL of LB agar solution (25 g of LB broth and 12 g of agar in 1 L of DI water). Simulated gastric fluid (SGF) and simulated intestinal fluid (SIF) were prepared as described in the United States Pharmacopoeia. Briefly, SGF was prepared by dissolving 2.0 g of NaCl and 3.2 g of pepsin in 1 L of DI water, and the pH was adjusted to 1.5 with HCl. The SGF was filtered by a 0.22 μm membrane before usage. SIF was prepared by dissolving 6.8 g of KH₂PO₄ and 10 g of trypsin in 1 L of DI water, and the pH was adjusted to 6.8 with NaOH. The SIF was filtered by a 0.22 μm membrane before usage.

### Synthesis of HA-PPS Conjugate

PPS. To a stirred solution of 100 μL (1.15 mmol) of 3-mercaptopropionic acid in 30 mL of anhydrous tetrahydrofuran was added 524 μL (3.45 mmol) of 1,8-diazabicyclo[5.4.0]-7-undecane (DBU) in an ice bath under stirring, and the reaction mixture was stirred for 30 min under a nitrogen atmosphere. Then, 1.9 mL (21.15 mmol) of propylene sulfide was added dropwise, and the reaction mixture was allowed to stir at 60 °C overnight. After that, the reaction was quenched by adding 5 mL of H₂O, purified by precipitation in cold methanol, and the solvent was evaporated under reduced pressure to produce PPS as a yellow oil. ¹H NMR (400 MHz, CDCl₃) δ 3.03-2.83 (m, CH₂), 2.72-2.56 (m, CH), 1.47-1.30 (d, CH₃); mass spectrum (ESI) m/z 1587.2 (M-H)— (C₆₃H₁₂₅O₂S₂₁ requires 1587.3).

PPS—NH₂. To a stirred solution of 158.6 mg (100 μmol) of PPS in 20 mL of dichloromethane was added 23 mg (200 μmol) of NHS and 48 mg (250 μmol) of EDCI, and the reaction mixture was stirred at room temperature for 30 min. Then, 133 μL (2 mmol) of ethylenediamine was added dropwise to the mixture, and stirring was continued overnight at room temperature. After that, the reaction mixture was diluted by adding 20 mL of dichloromethane, washed successively with H₂O and brine, dried over MgSO₄, and filtered. The solvent was concentrated under reduced pressure to give PPS—NH₂, which was used directly without further purification.

HA-PPS conjugate. Before synthesizing the HA-PPS conjugate, the acid form of HA was prepared from HA sodium salt. Briefly, HA sodium salt was dialyzed in 0.01 M HCl solution overnight and then lyophilized to produce the acid form of HA. 100 mg of HA was dissolved in 10 mL of H₂O, and 7 mg (60 μmol) of NHS and 14.5 mg (75 μmol) of EDCI were added. The mixture was stirred for 30 min at room temperature. Then, a solution containing 40 mg of PPS—NH₂ in 1 mL of tetrahydrofuran was added dropwise, and the stirring was continued for another 24 h at room temperature under nitrogen protection. After that, the mixture was dialyzed against water/methanol at a ratio of 1:1 three times for one day, followed by distilled water three times for one day. The solvent was removed by lyophilization to generate the HA-PPS conjugate. ¹H NMR (400 MHz, D₂O/Acetone-D6 1:1) δ 4.76-4.56 (m, 252H), 4.10-3.89 (m, 639H), 3.83-3.62 (m, 628H), 3.02-2.88 (m, 26H, CH₂ on PPS), 2.72-2.64 (m, 13H, CH on PPS), 2.43-2.33 (m, 372H, CH₃ on HA), 1.56-1.35 (m, 39H, CH₃ on PPS).

### Synthesis of the ROS-Responsive Linker.

2,2,2-Trifluoro-N-(2-mercaptoethyl)acetamide (2). To a stirred solution of 5 g (64.8 mmol) of cysteamine in 100 mL of methanol was added 9.3 mL (77.8 mmol) of ethyl trifluoroacetate and 13.5 mL (97.2 mmol) of triethylamine, and the reaction mixture was stirred at room temperature overnight. After that, the solvent of methanol was evaporated under reduced pressure, and 100 mL of dichloromethane was added. The mixture was then washed with water and brine, dried over MgSO₄, filtered, and concentrated under reduced pressure to give the crude product. The residue was purified by chromatography on a silica gel column to afford compound 2: yield 7.6 g (68%); silica gel TLC Rr 0.5 (5:1 hexane-EtOAc); ¹H NMR (400 MHz, CDCl₃) δ 6.81 (s, 1H), 3.56 (q, J=6.3 Hz, 2H), 2.82-2.70 (m, 2H), 1.42 (t, J=8.6 Hz, 1H).

N,N′-((Propane-2,2-diylbis(sulfanedlyl))bis(ethane-2,1-diyl))bis(2,2,2-trifluoroacetamide) (3). To a stirred solution of 7.5 g (43.4 mmol) of compound 2 in 100 mL of dry tetrahydrofuran was added 900 mg (5.22 mmol) of p-toluenesulfonic acid at room temperature under nitrogen protection, and the reaction mixture was stirred for 10 min. Then, 50 g of molecular sieves were added, and the reaction mixture was stirred for an additional 30 min. Next, 1.25 g (17.4 mmol) of 2-methoxypropene was added to the reaction mixture, and the stirring was continued for 24 h at room temperature. After that, the tetrahydrofuran solvent was evaporated under reduced pressure, and 100 mL of dichloromethane was added. The mixture was then washed with water and brine, dried over MgSO₄, filtered, and concentrated under reduced pressure. The residue was purified by chromatography on a silica gel column to give the compound 3: yield 3.1 g (37%); silica gel TLC Rr 0.5 (3:1 hexane-EtOAc); ¹H NMR (400 MHz, CDCl₃) δ 6.84 (s, 2H), 3.60 (q, J=6.5 Hz, 4H), 2.85 (t, J=6.7 Hz, 4H), 1.63 (s, 6H).

2,2′-(Propane-2,2-diylbis(sulfanedlyl))bis(ethan-1-amine) (ROS-responsive linker). 3.0 g (7.8 mmol) of compound 3 was dissolved in 20 mL of NaOH (6 M), and the reaction mixture was stirred at room temperature for 4 h. Then, the reaction mixture was extracted by dichloromethane, washed with brine, dried over MgSO₄, filtered, and concentrated under reduced pressure to give the pure ROS-responsive linker quantitatively: yield 1.5 g; ¹H NMR (400 MHz, CDCl3) δ 2.92 (t, J=6.5 Hz, 4H), 2.74 (t, J=6.5 Hz, 4H), 1.62 (s, 6H), 1.35 (s, 4H).

Preparation and characterization of HPN. HA-PPS conjugate was dissolved in distilled water, and ultrasonication (in intervals of 2 s on and 2 s off) was performed for 10 min in an ice bath to prepare HPN nanoparticles. The nanoparticle size and zeta potential of HPN were measured by dynamic light scattering (DLS). The morphologies of HPN were visualized by transmission electron microscopy (TEM, FEI Tecnai T12). Briefly, a drop of HPN solution was deposited onto a carbon-coated copper grid. After the sample completely dried, the sample was imaged by TEM.

ROS-scavenging activity of HPN. The ROS-scavenging activity of HPN was determined by measuring the fluorescent signals of DCF oxidized from DCFDA by peroxy radicals in the presence of HPN. PBS and PPS were used as the control groups. Briefly, 50 μM of DCFDA in PBS was incubated with 1 mM of H₂O₂ in the presence of PBS, PPS (80 μg/mL), and HPN (1 mg/mL) at 37 °C. The fluorescent signals were monitored using a microplate reader (Infinite M Plex, Tecan) for 1 h (excitation: 490 nm and emission: 520 nm).

Resistance of HPN against hyaluronidase-mediated degradation. The degraded product of HA by hyaluronidase, N-acetyl-D-glucosamine, was measured by a colorimetric method to assess the hyaluronidase-mediated degradation of HPN. Briefly, 1 mg/mL of HA and HPN were separately incubated with 150 U/mL of hyaluronidase in pH 6.0 PBS buffer at 37 °C. At predetermined time points, 100 μL of the samples were taken and diluted by 400 μL of PBS. The mixture was kept at 100 °C for 5 min to stop the enzymatic reaction. Next, 100 μL of 0.8 M potassium tetraborate (pH 9.0) was added to the mixture. After being kept at 100 °C for 3 min, the mixture was cooled in water, and 3 mL of p-dimethylaminobenzaldehyde (DMAB) reagent (10 g of DMAB dissolved in 100 mL of acetic acid containing 12.5% of 10 M HCl) was added. The OD_{544 nm} values were measured in 5 min by a microplate reader to evaluate the level of hyaluronan breakdown product.

Strains and culture conditions. The strains Escherichia coli Nissle 1917 (EcN), Escherichia coli K12 (E. coli K12), and Escherichia coli (Migula) Castellani and Chalmers (E. coli CC) were used in this Example. The bacterial cells were cultured on a LB agar plate (1.5% agar). Before each experiment, the cells were cultured in liquid LB medium overnight at the shaking speed of 225 rpm at 37 °C.

Cytoprotective effect of HPN against ROS-induced damage. The protective effect of HPN against ROS-induced damage was measured in colonic epithelial cells (HCT116) and bacteria (EcN and E. coli K12), respectively, using a cell counting kit-8 (CCK-8) assay. Briefly, for the colonic epithelial cells, HCT116 cells (8×10³ per well) were cultured in a 96-well plate for 24 h at 37 °C. Then, the culture medium was removed, and 100 μL of fresh medium and fresh medium containing 100 μM of H₂O₂ with or without HPN (0.5 mg/mL) was added to each well. After incubation for 72 h, the cells were washed with culture medium, and then fresh medium (100 μL) was added to each well, followed by the addition of 10 μL of CCK-8 solution. After incubation for 1 h at 37 °C, the OD_{450 nm} values were measured using a microplate reader. The cell viability was normalized to HCT116 cells without H₂O₂ and HPN treatment.

For bacterial strains, 100 μL of EcN and E. coli K12 in LB medium or LB medium containing H₂O₂ (100 μM) with or without HPN (0.5 mg/mL) were separately seeded into a 96-well plate with an initial OD_{600 nm} value ~0.15, and incubated for 12 h. Afterward, 10 μL of CCK-8 solution was added to each well and incubated for an additional 1 h. The OD values were recorded at 450 nm by a microplate reader, and the viability was normalized to bacteria without H₂O₂ and HPN treatment.

Encapsulation of EcN with the NE layer and characterization of NE-EcN. The EcN cells, picked from an LB agar plate, were cultured in LB medium at 37 °C overnight. After the EcN cells were washed with PBS twice, 0.5 mg/mL of NE solution in PBS was added and incubated for 3 h at a shaking speed of 200 rpm. The formed NE-EcN cells were collected by centrifugation after being washed with PBS three times to remove residual NE molecules. The formed NE layer on the bacterial surface was characterized by TEM. Briefly, a drop of NE-EcN solution in DI water was deposited onto a carbon-coated copper grid. After completely drying, the sample was imaged by TEM. Moreover, the sizes and zeta potentials of EcN and NE-EcN were determined by DLS.

External environment resistance assay for NE-EcN. The protective effects of the NE layer on EcN against simulated gastrointestinal conditions, including SGF (pH=1.5) supplemented with pepsin (0.32%), SIF (pH=6.8) supplemented with trypsin (10 mg/mL), and bile salts (0.4%), were measured. Briefly, for the SGF resistance assay, equal amounts of EcN and NE-EcN were separately subjected to SGF and incubated at 37 °C with a shaking speed of 225 rpm. At predetermined time points, 50 μL of the sample was taken, washed with PBS, and spread on LB agar plates in sequential 10-fold dilutions. The colonies were counted after 24 h of incubation at 37 °C. For resistance against SIF and bile salt, equal amounts of EcN and NE-EcN were separately subjected to SIF and bile salts in LB medium. At predetermined time points, the samples were collected and washed with PBS. After that, the samples were resuspended in 100 μL of PBS, followed by adding 10 μL of CCK-8 solution, and incubated for 1 h at 37 °C. The OD_{450 nm} values were recorded to evaluate cell viability.

Preparation and characterization of HPN-NE-EcN. To prepare the ROS-responsive linker bound to HPN, the HA-PPS molecule was first reacted with sulfo-NHS to produce HA-PPS—NHS. Briefly, to a stirred solution of 20 mg of HA-PPS in 10 mL of distilled water was added 10 mg (0.05 mmol) of EDCI and 4 mg (0.02 mmol) of sulfo-NHS, and the reaction mixture was stirred for 4 h at room temperature to generate HA-PPS—NHS. After dialysis to remove residual EDCI and sulfo-NHS, the solution of HA-PPS—NHS was ultrasonicated to prepare the sulfo-NHS exposed HPN nanoparticle, which was then reacted with the ROS-responsive linker (20 mg, 0.1 mmol) at room temperature overnight to prepare the ROS linker exposed HPN. The generated HPN was collected by centrifugation and washed with water three times to remove the residual ROS linker. Next, the EcN cells were incubated in 0.5 mg/mL of the NE solution in PBS for 1 h at a shaking speed of 200 rpm. Then, the ROS linker exposed HPN was added, and the mixture was incubated for an additional 2 h. The generated HPN-NE-EcN was collected by centrifugation at 4000 rpm and washed with water three times to remove the residual HPN nanoparticles.

The formed HPN-NE-EcN was first characterized by TEM images. Briefly, the HPN-NE-EcN solution in distilled water was deposited on the surface of the carbon-coated copper grid. After completely drying, one droplet of UranyLess was dropped on the grid surface and kept for 3 min. Then, the grid was washed with distilled water and dried in the air. Next, the dried grid was placed on a 3% lead citrate droplet for 3 min. After washing in distilled water, the grid was dried in the air, and images were taken by TEM. The HPN-NE-EcN was further characterized by laser scanning confocal microscopy (LSCM, Nikon A1RS). To be imaged by LSCM, HA-PPS was first labeled with FAM fluorescence before making HPN-NE-EcN. Briefly, HA-PPS was dissolved in water. NHS and EDCI were added to the mixture, and the mixture was stirred at room temperature for 15 min. Then, FAM-NH₂ was added, and the stirring was continued for 4 h. After that, the mixture was dialyzed in water for 2 days to remove the residual FAM fluorescence. Next, the FAM-labeled HA-PPS was used to prepare the HPN-NE-EcN as described above, and the resulting HPN-NE-EcN was imaged by LSCM. Moreover, the loading efficacy of the HPN on the surface of the bacteria was quantified by fluorescence measurement. Briefly, the concentration of HPN correlated with fluorescence was quantified, and a standard curve was created. After preparing the HPN-NE-EcN, the amount of HPN conjugated on the bacterial surface was quantified according to a standard curve.

Measurement of growth curves of EcN, NE-EcN, and HPN-NE-EcN. To assess whether the NE layer and HPN nanoparticles on the surface of EcN would affect the growth and proliferation of EcN, the growth curves of EcN, NE-EcN, and HPN-NE-EcN were measured. Briefly, after encapsulating EcN with the NE layer and conjugating HPN on the surface of NE-EcN, the bacteria were diluted and seeded into a 96-well plate with an OD₆₀₀ value ~0.15 and incubated at 37 °C with gentle shaking. The OD₆₀₀ values were monitored every 0.5 h for 12 h in total by a microplate reader. Uncoated EcN was used as a control.

Electrotransformation of EcN with PAKgfpLux2. The EcN cells were electrotransformed with a fluorescent reporter plasmid, PAKgfpLux2, for IVIS imaging to monitor the distribution of bacteria in vivo. Briefly, EcN cells (1×10⁹ CFU) were collected and washed with distilled water three times to remove ions from PBS. Then, the EcN cells were resuspended in 40 μL of distilled water and mixed with 1 μL of the PAKgfpLux2 plasmid at a final concentration of 1 μg/mL. The mixture was kept on ice for 1 min before being transferred to a pre-chilled electroporation cuvette (0.1 cm), and the bacterial suspension was tapped to the bottom of cuvette to eliminate bubbles. Next, the cuvette was placed into a chamber. After being pulsed once (1.8 kV, 4 milliseconds, MicroPulser, BIO-RAD), the cuvette was removed from the chamber, and 1 mL of LB medium was added immediately. The cell suspension was then transferred to a cell culture tube and incubated at 37 °C for 1 h. Afterward, the bacteria were collected by centrifugation and spread on an LB agar plate containing 100 μg/mL of ampicillin for selection and incubated at 37 °C overnight.

Evaluation of the adhesive effect of HPN-NE-EcN in vivo. The animal study protocol was approved by the Institutional Animal Care and Use Committee (IACUC) at the University of Wisconsin-Madison. To investigate the adhesive effect of HPN-NE-EcN in the intestines, the EcN cells were initially electrotransformed with the PAKgfpLux2 plasmid as described above. Then, the EcN cells were encapsulated with the NE layer and further conjugated with HPN on the bacterial surface. The generated HPN-NE-EcN was orally administered to mice, and the distribution of EcN was monitored through bioluminescence signals via an IVIS. Briefly, mice (female, 6-8 weeks old) were randomly assigned to three groups (n=3 in each group). Before administration of the EcN formulations, the mice were fasted for 18 h, but were given drinking water containing IPTG (5 g/L) and ampicillin (1 g/L). Moreover, the bioluminescence of EcN cells was induced by IPTG (1 mM) for 4 h before administration. Then, 100 μL of EcN, NE-EcN, and HPN-NE-EcN (bacteria dose: 1×10⁸ CFU) in PBS was orally administered, respectively. Afterward, the mice were given normal chow with IPTG and ampicillin in the drinking water. At 4 h, 12 h, 24 h, and 48 h, bioluminescence in the mice was detected by an IVIS. Furthermore, the mice were sacrificed after 48 h of receiving the different EcN formulations, and the GI tracts were isolated and imaged.

To visualize HPN nanoparticles in mice, the HPN was labeled with Alexa fluor 647 before preparing HPN-NE-EcN. Briefly, 15 mg of prepared HA-PPS—NHS was dissolved in 10 mL of distilled water, and 0.3 mg of Alexa fluor 647-NH₂ was added to the solution. The reaction mixture was stirred overnight to generate the Alexa fluor 647-labeled HA-PPS molecule, which was dialyzed in distilled water for 2 days to remove the residual free fluorophore. The fluorescence-labeled HA-PPS was used to prepare the HPN and HPN-NE-EcN for visualizing the HPN in vivo. After receiving the HPN and HPN-NE-EcN (7.5 mg/kg) for 4 h and 12 h, the mice were imaged via IVIS to visualize the HPN.

The fluorescent signals of EcN cells and HPN nanoparticles in the intestines were also imaged by LSCM. Briefly, EcN cells were labeled with FAM, and the HPN nanoparticles were marked by Alexa fluor 647. Then the HPN-NE-EcN was prepared using FAM-labeled EcN and Alexa fluor 647-labeled HPN. After 48 h of receiving EcN and HPN-NE-EcN (bacteria dose: 1×10⁸ CFU), the mice were sacrificed, and the colon tissues were isolated for frozen slides preparation. The frozen slides were then fixed, dehydrated, and imaged by LSCM to visualize the fluorescence of EcN. For visualizing the HPN nanoparticles, the mice were given HPN and HPN-NE-EcN (7.5 mg/kg), respectively. After 12 h, the mice were sacrificed, colon tissues were isolated, and the frozen slides of colon tissues were prepared. Then, the slides were imaged by LSCM.

Prophylactic efficacy of HPN-NE-EcN against DSS-induced colitis. The prophylactic efficacy of HPN-NE-EcN against GI tract-related diseases was evaluated in a DSS-induced mouse colitis model. Briefly, mice (female, aged 6-8 weeks) were randomly divided to six groups: PBS-DSS⁻, PBS-DSS⁺, EcN, NE-EcN, HPN, and HPN-NE-EcN (n=6). The mice were given various EcN and HPN formulations (bacteria dose: 1×10⁸ CFU; HPN: 30 mg/kg) based on their group assignment via oral gavage every two days throughout the experimental period. 3% of DSS was added to the drinking water for the first 7 days to induce colitis except for the PBS-DSS⁻ group. After 7 days, DSS was removed, and all mice were given normal drinking water. On day 11, the mice were sacrificed, and the colon length was measured. A few major organs, including heart, liver, spleen, lungs, and kidneys, were harvested and fixed in 4% paraformaldehyde for histological analysis. Moreover, colon tissues were collected and separated into several sections for further analysis. In addition, mouse body weight was recorded daily.

Therapeutic efficacy of HPN-NE-EcN against DSS-induced colitis. Firstly, colitis was induced in the mice, and the mice were given different formulations of HPN and EcN to evaluate the therapeutic efficacy of HPN-NE-EcN against colitis. Briefly, the mice were randomly assigned to six groups (PBS-DSS⁻, PBS-DSS⁺, EcN, NE-EcN, HPN, and HPN-NE-EcN) with six mice in each group. The mice were given drinking water containing 3% DSS for 7 days to induce colitis. Afterward, the mice were given normal drinking water, and various HPN and EcN formulations (bacteria dose: 1×10⁸ CFU; HPN: 30 mg/kg) were administered for 4 days. After that, the mice were sacrificed, and the colon length was measured. Colon tissues were harvested and separated into several sections for further analysis. Moreover, the body weights for all the mice were recorded daily.

Histopathology studies. The histopathology analysis for evaluating the extent of colon damage was performed according to standard procedures for paraffin embedding and hematoxylin and eosin (H&E) staining. Briefly, the colonic tissues were fixed in 4% paraformaldehyde solution, embedded in paraffin, sectioned (4 μm), and stained with H&E. The resulting slides were scanned using a Nikon intensilight fluorescence microscope. Each section was scored blindly by a trained pathologist for histological evidence of colon damage by DSS with a scoring system described in Table 1. The scoring system presented in the supporting information included the severity of inflammation, depth of injury, extent of crypt damage, and percentage of colon tissue involvement.

Myeloperoxidase (MPO) assay. Colonic MPO, a marker for neutrophil infiltration, was measured to evaluate the level of inflammation in colon tissues. Briefly, the colon tissues were isolated from mice in prophylactic and therapeutic experiments and were weighed and washed thoroughly with PBS to clean the fecal matter. Next, the samples were homogenized in 0.5% of hexadecyltrimethylammonium bromide (5-fold v/m) in pH 6.0 PBS, freeze-thawed three times, and sonicated for 10 s to give a homogenous tissue suspension. Afterward, the clear supernatant was collected by centrifugation at a speed of 20000 g at 4 °C for 20 min. Subsequently, 50 μL of supernatant was added to a 96-well plate, and 200 μL of 1 mg/mL of dianisidine dihydrochloride containing 0.005% v/v H₂O₂ in pH 6.0 PBS was added. The plate was incubated for 20 min at room temperature. OD_{450 nm} values were measured to represent the MPO expression levels.

Evaluate ROS levels in colon tissues. The ROS-scavenging capability of HPN-NE-EcN was further evaluated in vivo. Briefly, colon tissues were immersed in 50 μM of DCFHDA solution in PBS immediately after being isolated from the mice in the prophylactic experiment and were incubated for 2 h at room temperature. Afterward, the colon tissues were washed with PBS three times to remove free DCFHDA, and frozen slides for the colon tissues were prepared. Next, the frozen slides were fixed in pre-cooled (−20 °C) acetone for 10 min. The acetone was then poured off. The slides were dried in the air for 20 min and rinsed with PBS twice for 5 min each time. Afterward, the slides were incubated in Hoechst 33342 solution with PBS for 10 min, and then rinsed with water four times to remove the residual Hoechst 33342. Next, the slides were dehydrated through 4 changes of ethanol (95%, 95%, 100%, and 100%) for 5 min each. After evaporation of ethanol, the slides were covered with a coverslip using Permount solution and imaged by LSCM.

Statistics. Statistical analysis was evaluated using GraphPad Prism 8. The statistical significance was determined using Student's t-test and one-way ANOVA analysis followed by Tukey's or Fisher's LSD multiple comparison. The differences between experimental and control groups were considered statistically significant at P<0.05. (ns) P>0.05, *P<0.05, **P<0.01, ***P<0.001.

### Results and Discussion

### Preparation and Characterization of HPN as the ROS Scavenger.

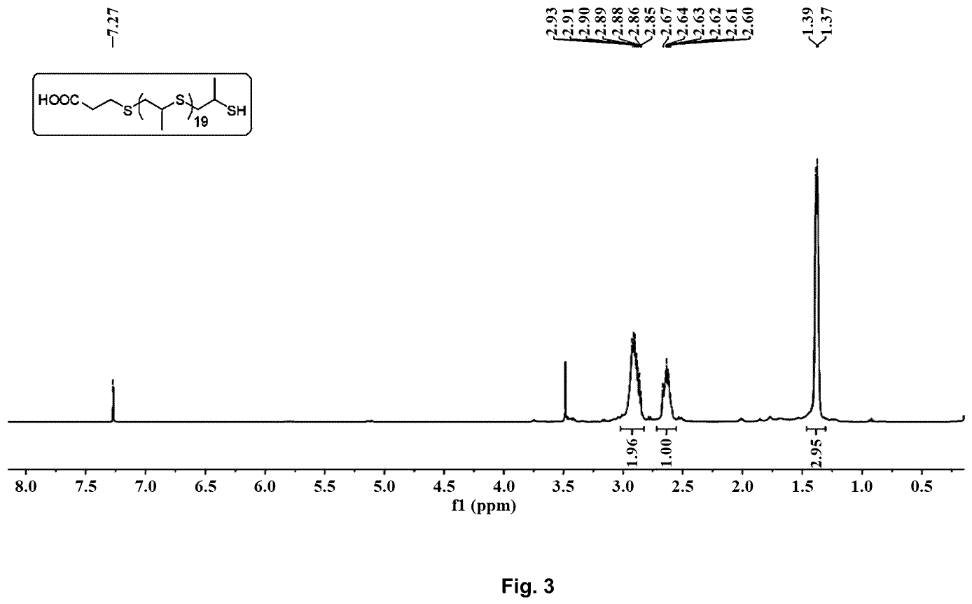

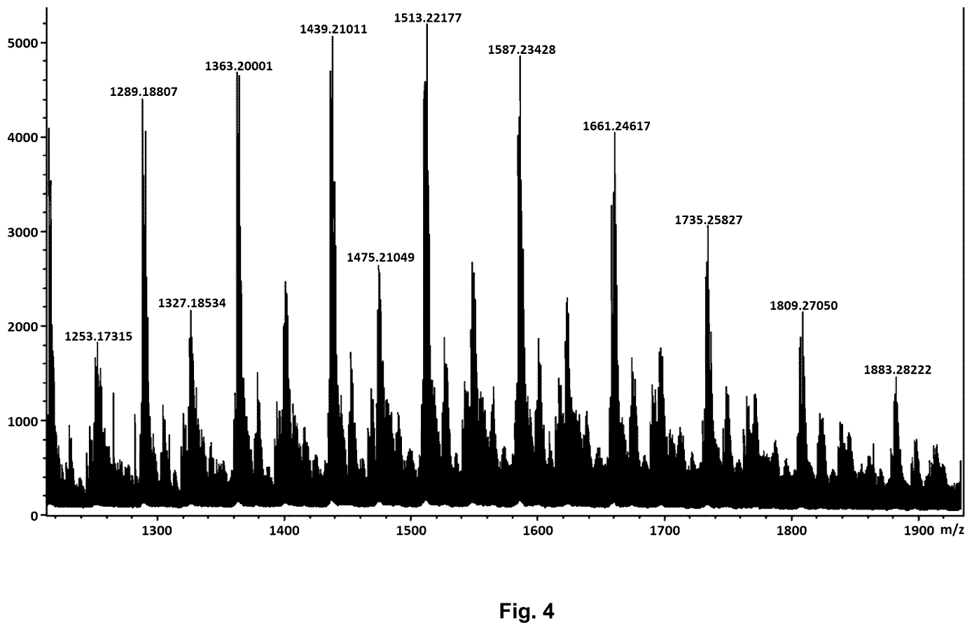

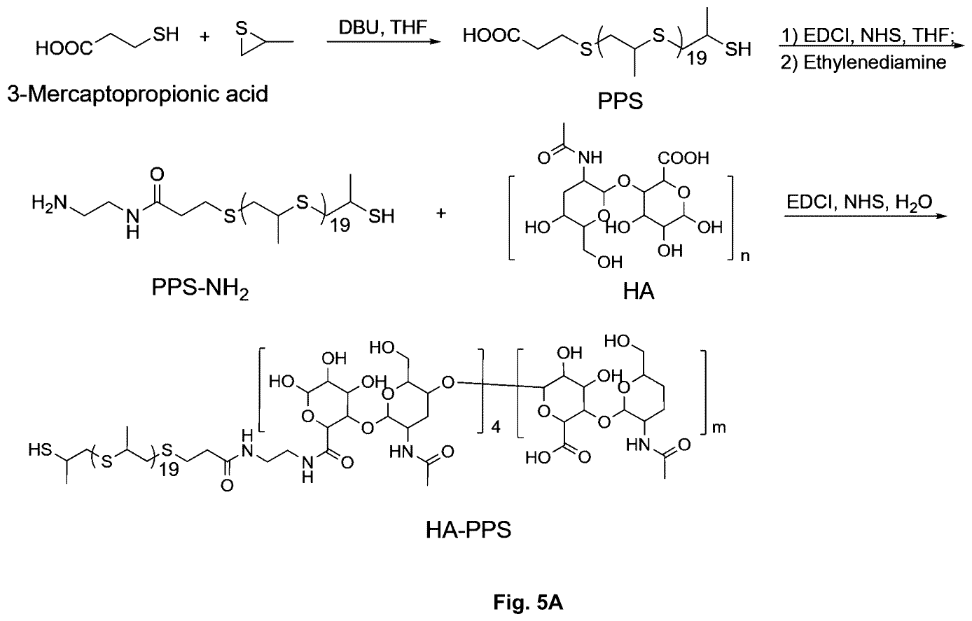

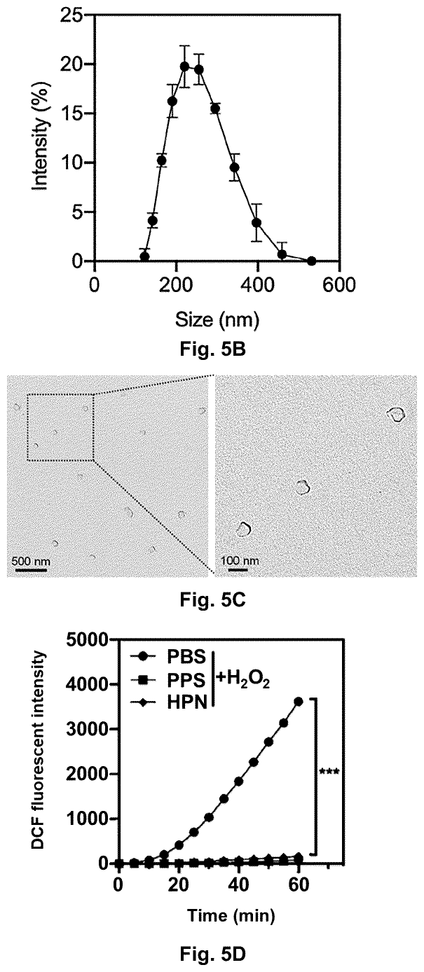

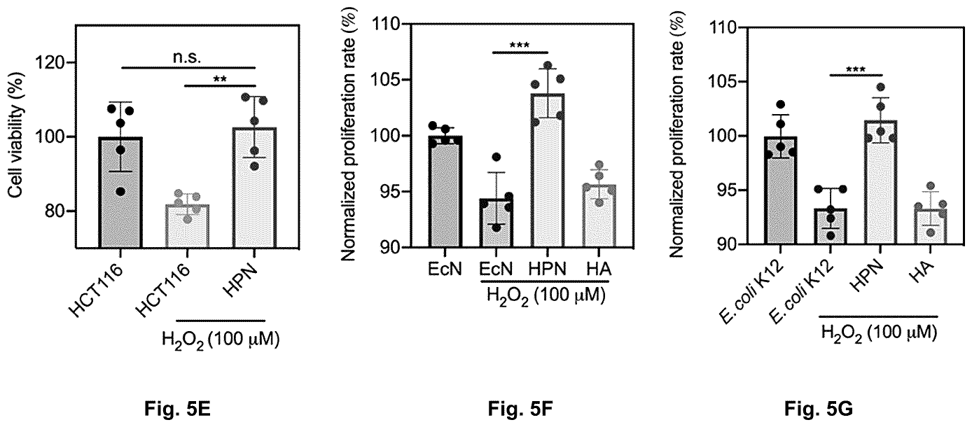

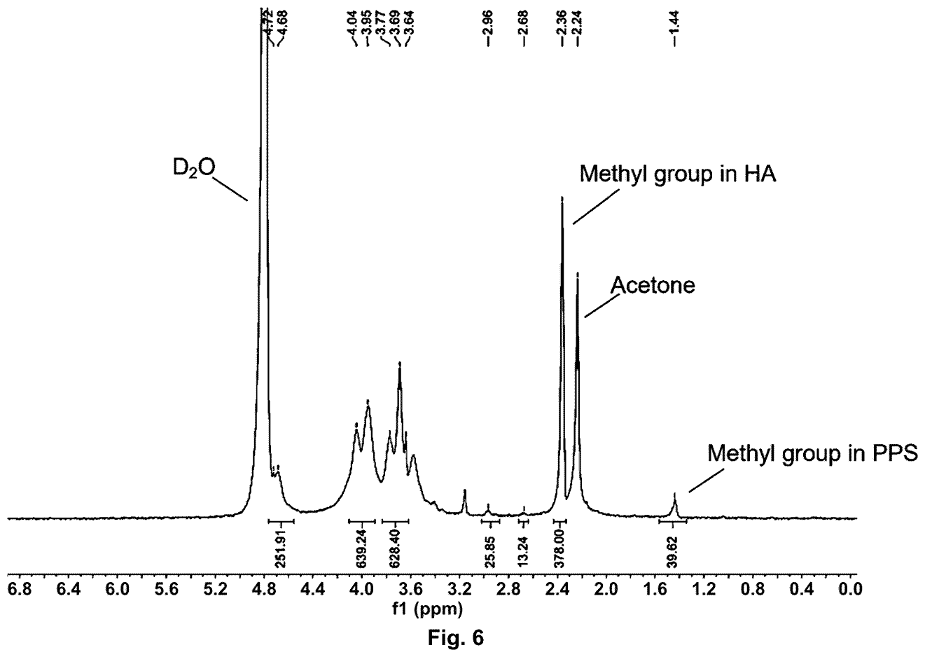

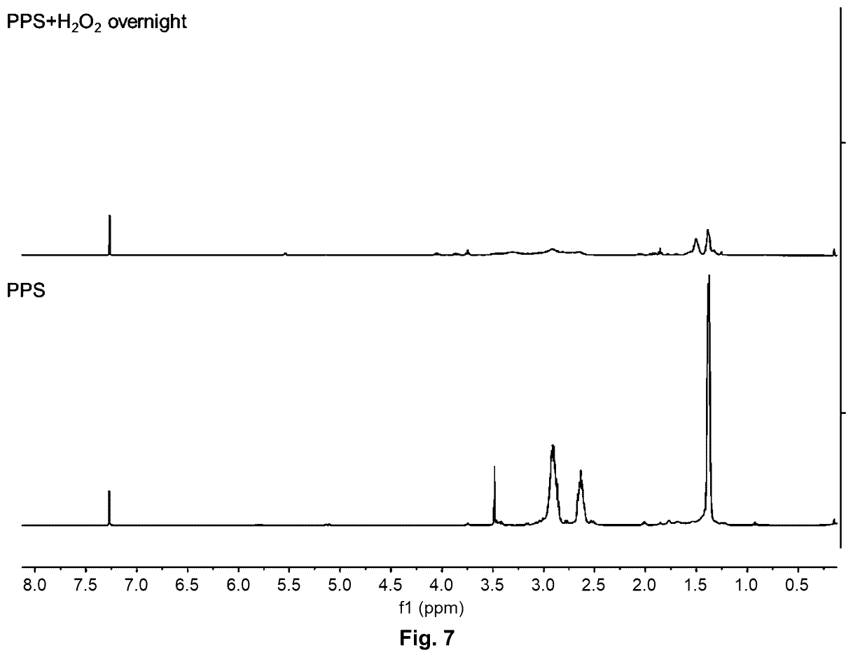

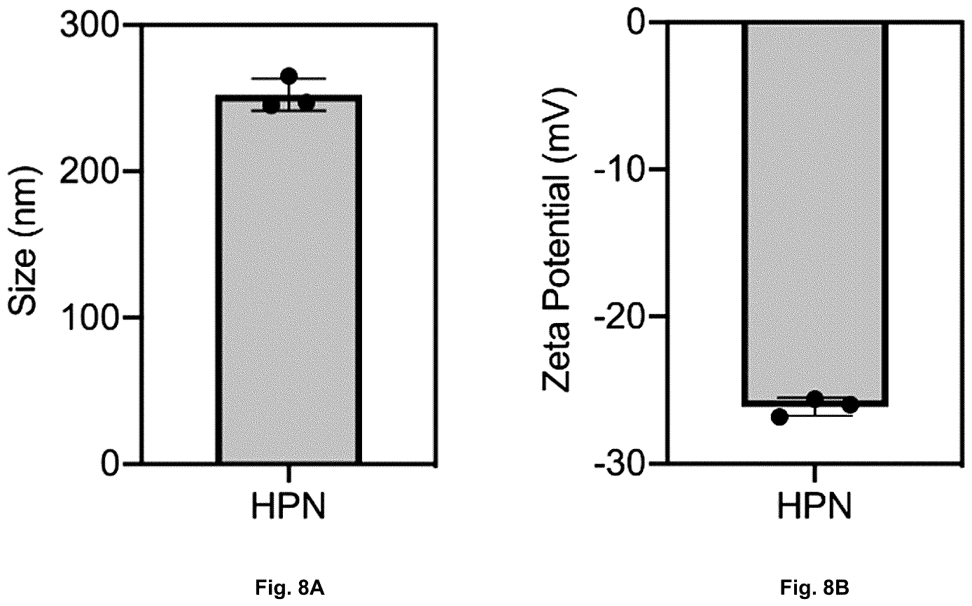

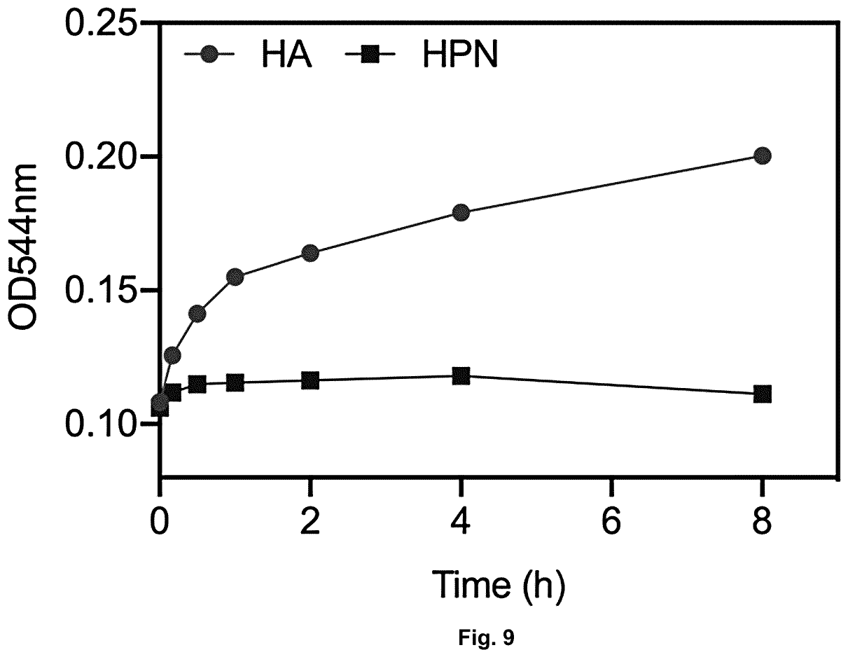

Firstly, we synthesized the PPS polymer with 3-mercaptopropionic acid and propylene sulfide as the starting materials in the presence of a strong base, DBU. The synthesized PPS had 20 units of propane-2-thiol, which was confirmed by nuclear magnetic resonance spectroscopy (NMR) and mass spectrometry (FIGS. 3-4). To conjugate HA with PPS, ethylenediamine was used as the linker. The carboxyl group of PPS was activated by NHS under EDCI conditions and reacted with ethylenediamine to generate PPS—NH₂, which was then amidated with the acid form of HA to prepare the HA-PPS conjugate (FIG. 5A). The structure of HA-PPS was confirmed by NMR. An average density of 2 molecules of PPS (~40 units of propane-2-thiol) was conjugated on each 100 kDa HA molecule (FIG. 6), suggesting a high loading efficacy. PPS could be oxidized in the presence of H₂O₂, indicating its ROS-scavenging activity (FIG. 7). The HA-PPS conjugate in aqueous buffer self-assembled to form HPN, which was first characterized by dynamic light scattering (DLS). The size of HPN was 245.2 nm (FIGS. 5B and 8A), and the zeta potential was −26.1 mV (FIG. 8B). Transmission electron microscopy (TEM) was used to visualize the morphology of HPN shown in FIG. 5C. HPN exhibited significant ROS-scavenging activity measured by dichlorofluorescein diacetate (DCFDA) fluorescence (FIG. 5D). DCFDA is oxidized in the presence of ROS, and this oxidation forms DCF which can be used as a fluorescent indicator. As a result, the fluorescent intensity is diminished in the presence of HPN. Due to the robust ROS-scavenging activity, HPN was able to protect human colonic epithelial cells (HCT116) against ROS-mediated cytotoxicity. As shown in FIG. 5E, the HPN-treated HCT116 cells displayed significantly higher viability than HCT116 cells after incubation with H₂O₂, and showed no significant difference compared to HCT116 cells without H₂O₂ treatment. Additionally, HPN conferred protection against ROS-mediated cytotoxicity for two common gut microorganisms, EcN (FIG. 5F) and Escherichia coli K12 (E. coli K12) (FIG. 5G). This result emphasizes the cytoprotective effect of HPN against ROS-induced cell damages. Interestingly, HPN was more resistant against hyaluronidase-mediated degradation compared to free HA (FIG. 9), likely due to the steric hindrance of the HPN nanostructure. The resistance to degradation might enhance its efficacy profile by preventing the quick clearance of the nanoparticle.

### Preparation and Characterization of NE-EcN and HPN-NE-EcN Against Environmental Assaults.

To specifically deliver HPN to regions of inflammation in the colon tissues, the HPN was conjugated onto the bacterial surface of EcN. Following oral administration, the EcN was able to deliver the ROS-scavenger to the affected areas due to the probiotic's colonic colonization effect. Moreover, this bacterial colonization effect is relevant because GI tract-related diseases are usually accompanied with a dysregulated microbiota. Previous research has shown that the addition of supplementary commensal bacteria is beneficial for regulating microbiota homeostasis in the GI tract for IBDs. Therefore, conjugating the ROS scavenger HPN onto the surface of probiotics has the potential to exhibit synergy for enhanced therapeutic efficacy in the setting of IBDs.

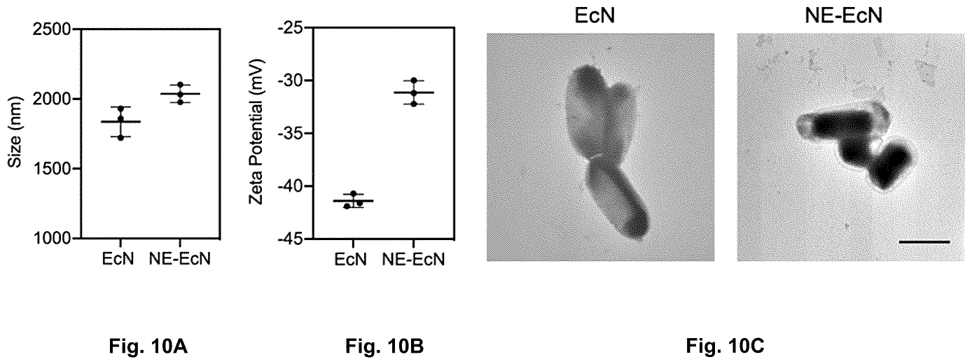

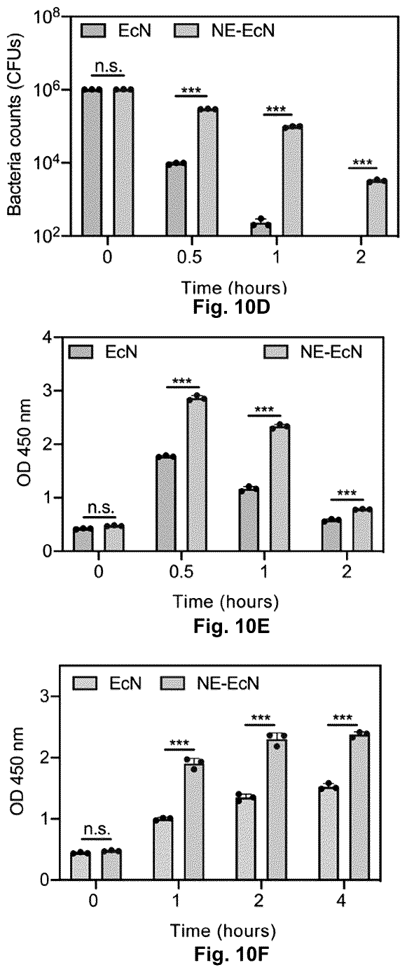

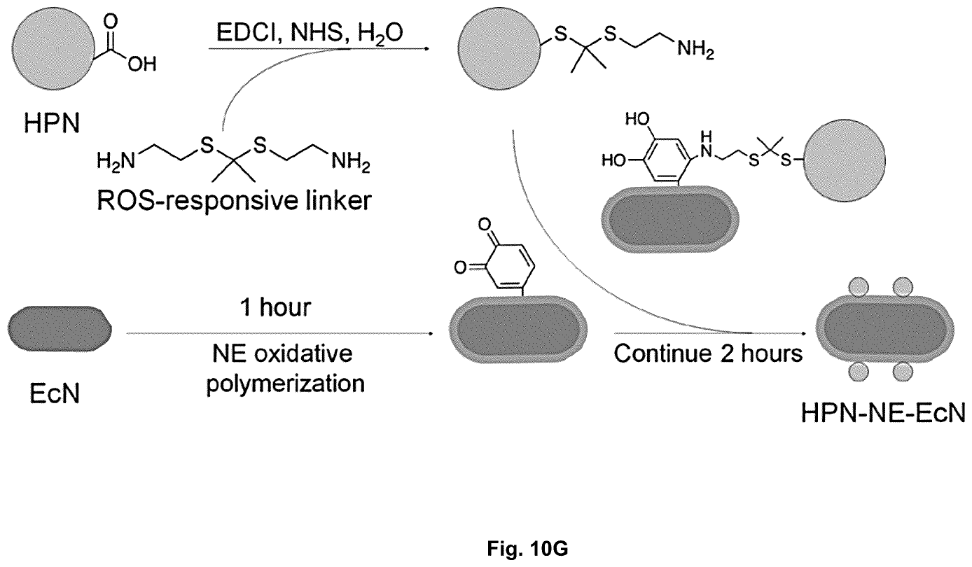

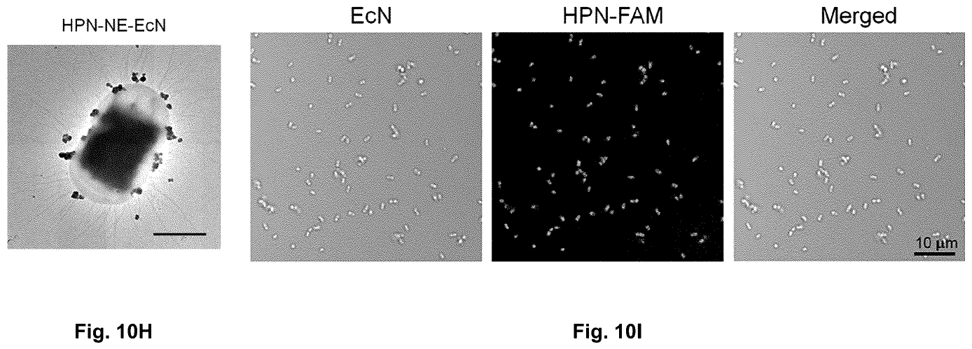

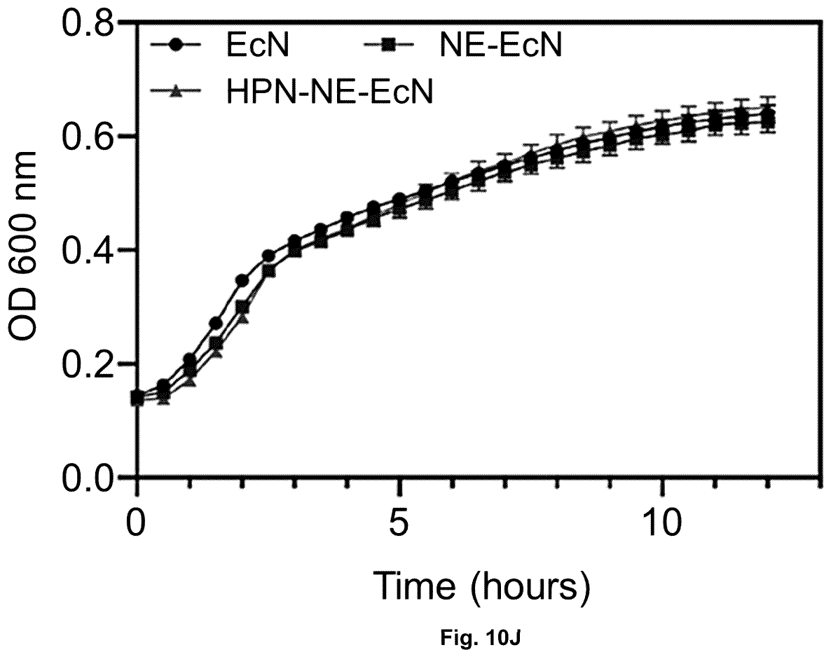

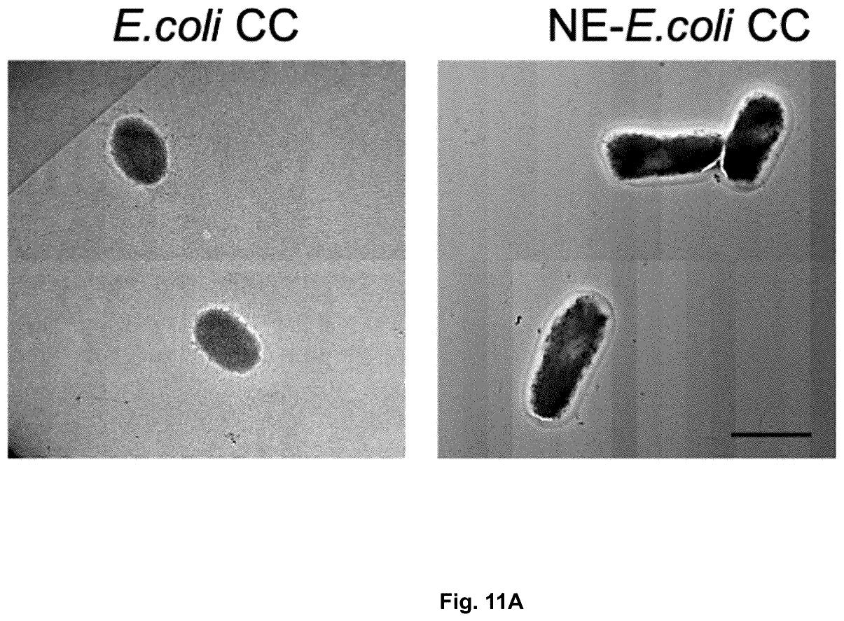

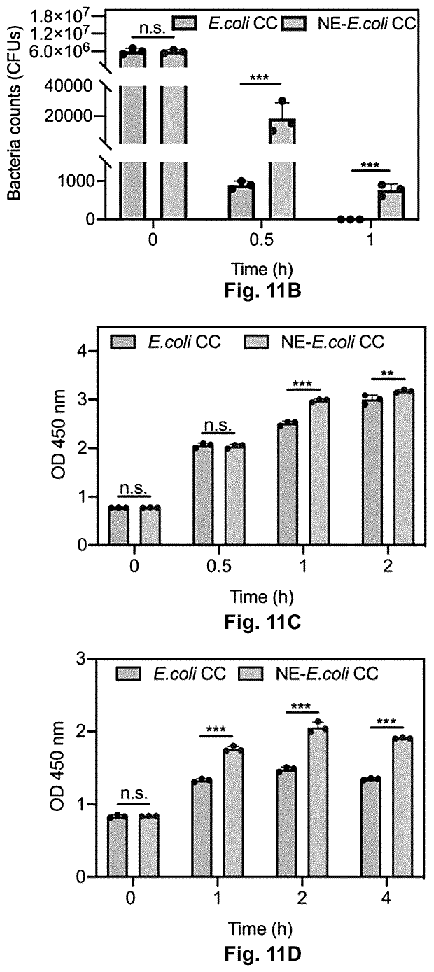

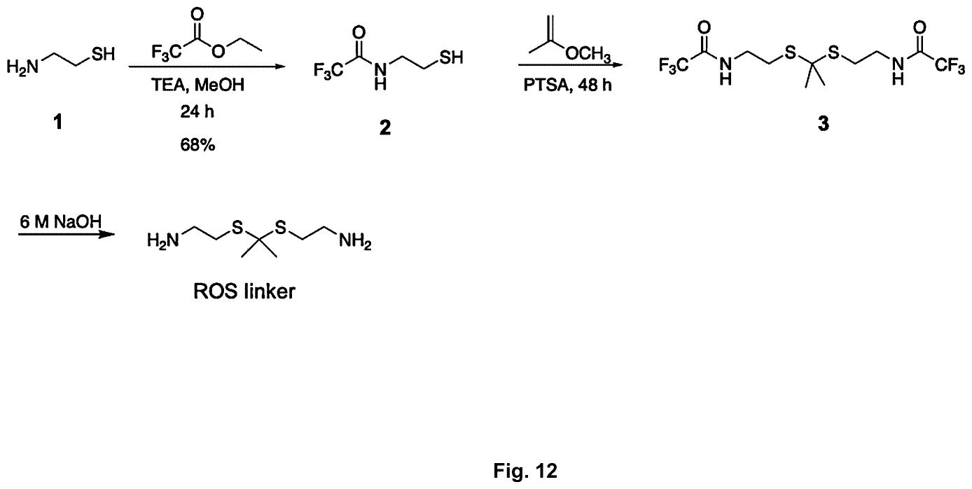

EcN, a well-known probiotic commercially used to modulate intestinal flora,²⁷ was chosen as the model bacterium in this Example. Firstly, EcN was encapsulated with a NE layer to protect the bacteria from the harsh environmental conditions presenting in the GI tract, and to extend the retention time of EcN in the intestines. After coating the bacteria with the NE layer, the size of EcN increased from 1837 nm to 2037 nm (FIG. 10A), and the zeta potential increased from −41.1 mV to −31.1 mV (FIG. 10B), indicating the existence of the NE layer on the surface of EcN. TEM images showed a transparent outer shell on the EcN (FIG. 10C), further demonstrating that the NE layer was formed and coated on the EcN surface. Next, we investigated whether the NE layer could protect EcN against environmental assaults. When EcN and NE-EcN were subjected to simulated gastric fluid (SGF) supplemented with pepsin that mimics the acidic gastric environment, the viability of NE-EcN was significantly higher than EcN after incubation for 0.5, 1, and 2 h (FIG. 10D). Notably, there were more than 3×10³ live bacteria in the NE-EcN group after 2 h incubation, whereas almost all uncoated EcN died. It was also found that NE-EcN exhibited enhanced survival rates in bile salts (FIG. 10E) and simulated intestinal fluid (SIF) containing trypsin (FIG. 10F) compared to uncoated EcN, demonstrating the protective effect of NE layer on EcN against external environment assaults. To verify the universality of the NE layer coating strategy for other live cells, we further coated the NE layer on another bacterial strain, Escherichia coli (Migula) Castellani and Chalmers (E. coli CC). As shown in TEM images in FIG. 11A, the NE layer was successfully coated on the surface of E. coli CC. Moreover, the NE layer offered protection for E. coli CC strain against external environmental assaults, including SGF (FIG. 11B), bile salt (FIG. 11C), and SIF (FIG. 11D). This finding supports the broad application of the NE layer coating strategy for bacterial protection in the gastrointestinal environment.

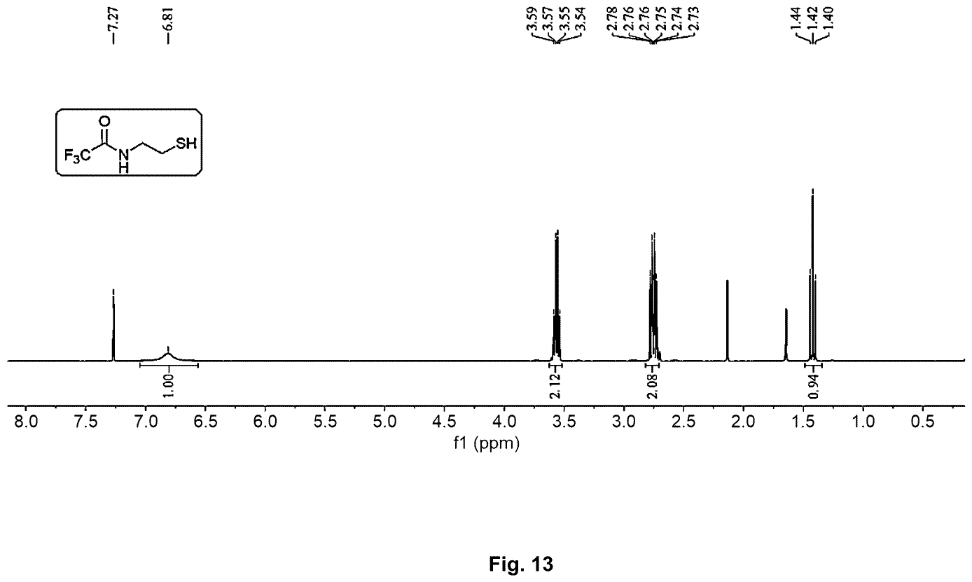

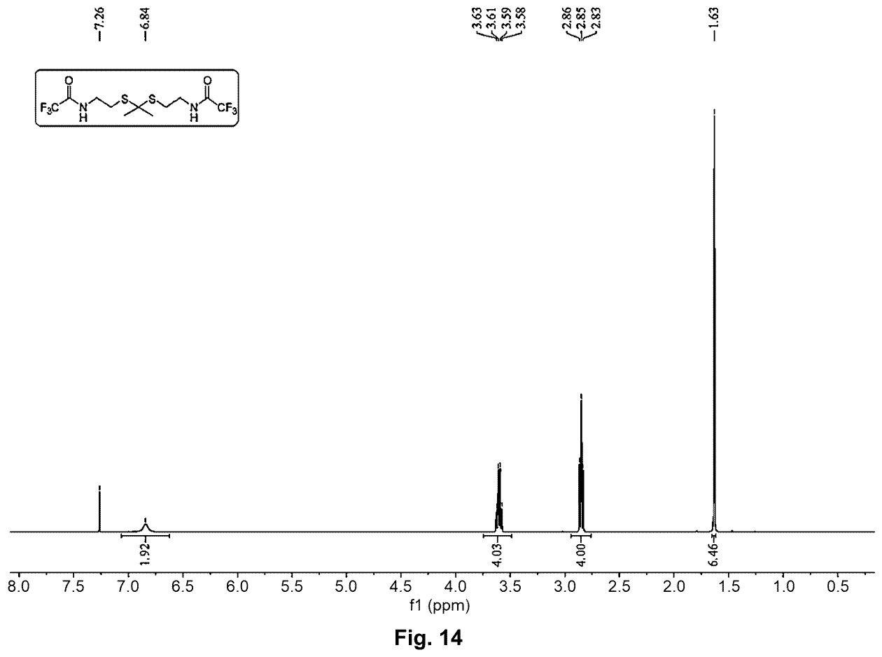

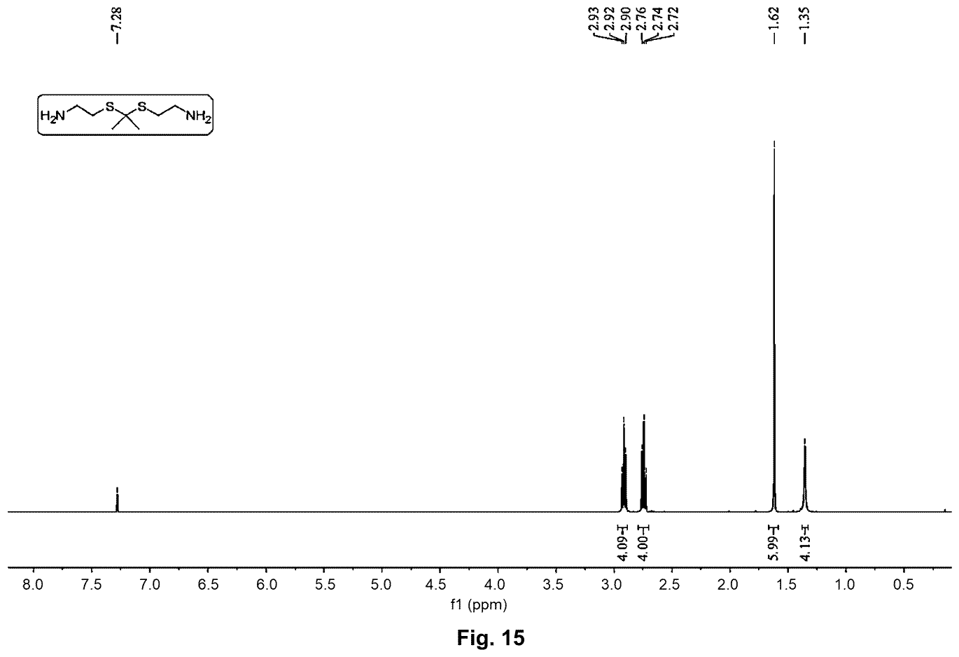

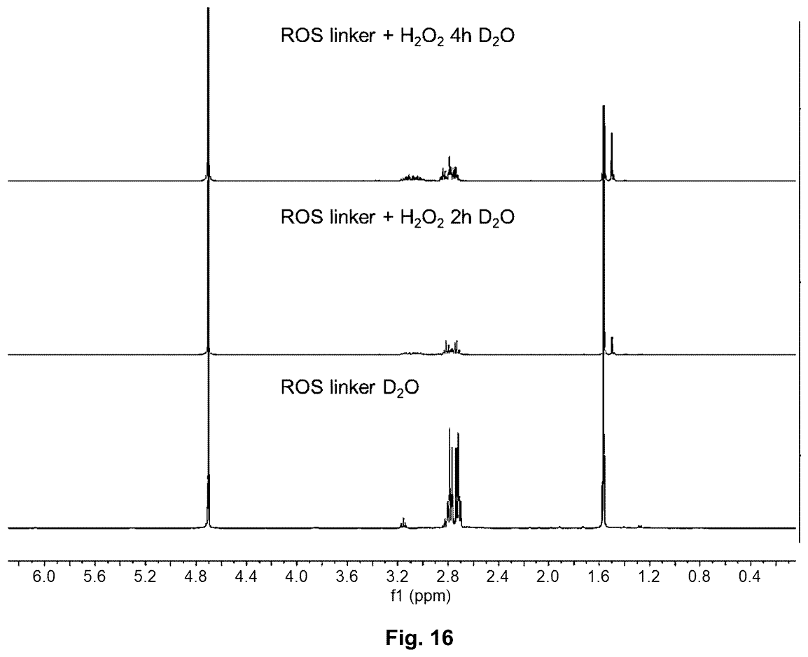

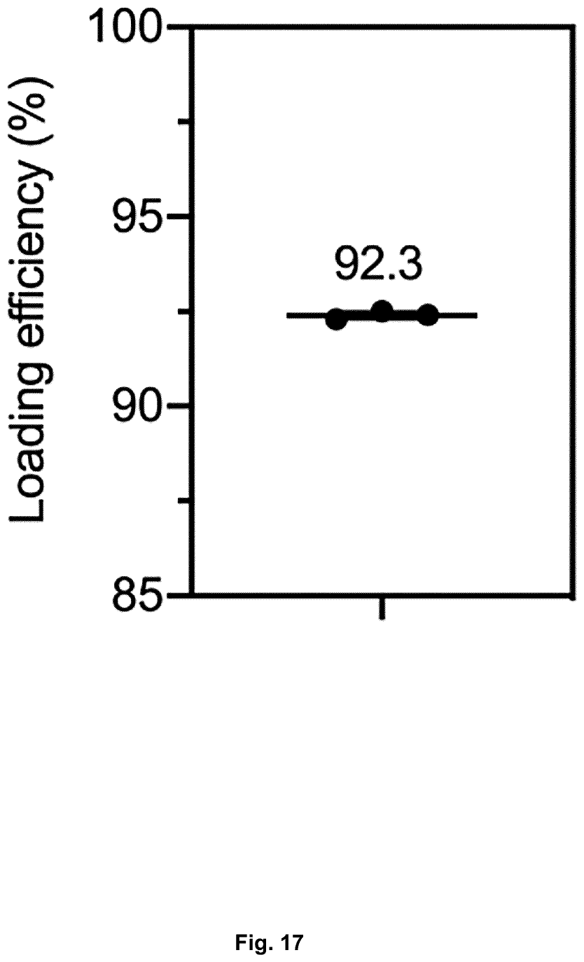

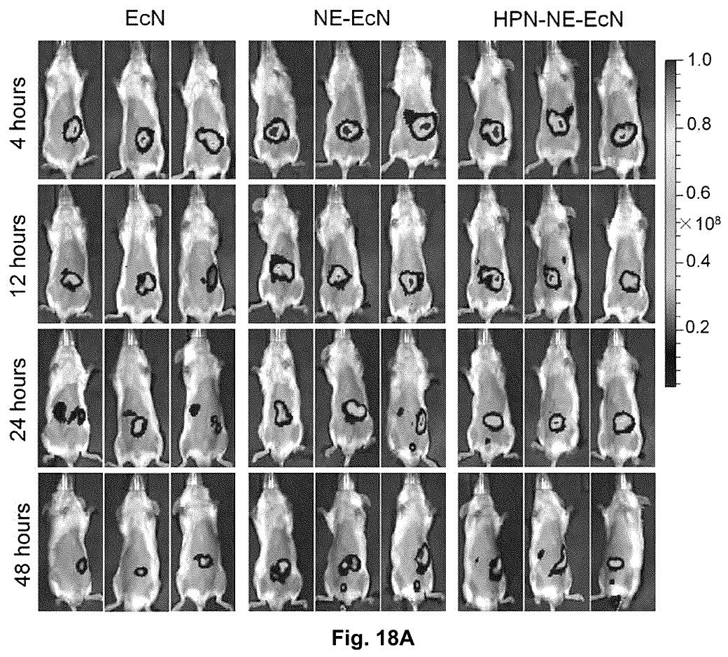

To conjugate HPN on the surface of NE-EcN, a ROS-responsive linker with a terminal amine group was synthesized²⁸. As shown in Fig. S9, the synthesis was started from cysteamine. The amine group of cysteamine was first protected with a trifluoroacetate group to generate intermediate 2, which was further reacted with 2-methoxypropene in the presence of p-toluenesulfonic acid to yield intermediate 3. Hydrolysis of compound 3 under sodium hydroxide gave the ROS-responsive linker. The structures of the intermediates and the ROS-responsive linker were confirmed by NMR (FIGS. 13-15). After incubation of the linker with H₂O₂ for 4 h, approximately 40% of the linker was degraded (FIG. 16), demonstrating that the linker was ROS responsive. The ROS-responsive linker will facilitate the release of HPN from EcN in the inflamed colon tissues with elevated ROS level. Next, the carboxyl groups of HPN were activated by NHS and then condensed with one of the amine groups from the linker. The other amine group exposed on HPN served as a reactant group for the auto-oxidation of NE to form the poly-norepinephrine film on the bacterial surface (FIG. 10G). The TEM image shown in FIG. 10H confirmed that HPN was successfully conjugated on the surface of the bacteria. The presence of HPN on the bacterial surface was further characterized by laser scanning confocal microscopy (LSCM), as evidenced by the FAM-labeled HPN observed on the surface of EcN (FIG. 10I). The coating efficacy of NE on the EcN was 92.3% (FIG. 17). To examine whether the NE coating and HPN conjugation affected the growth and proliferation of EcN cells, EcN, NE-EcN, and HPN-NE-EcN were each incubated in lysogeny broth (LB) medium, respectively. The growth curves were monitored for 12 h via OD_{600 nm} values. As shown in FIG. 10J, a similar growth rate was found among the three groups, demonstrating that the NE coating and conjugated HPN had a negligible impact on the growth and proliferation of EcN in vitro.

### Mucoadhesive Capability of HPN-NE-EcN.

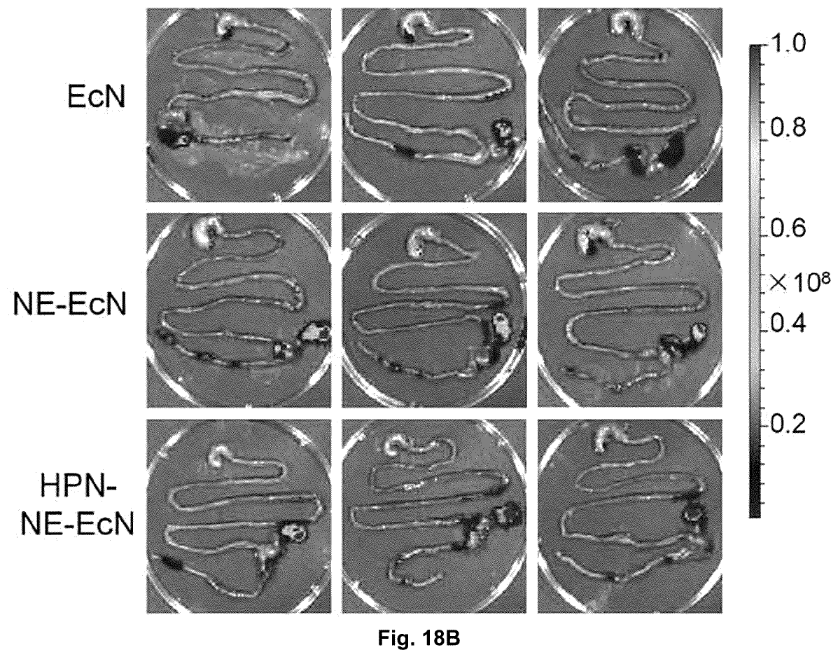

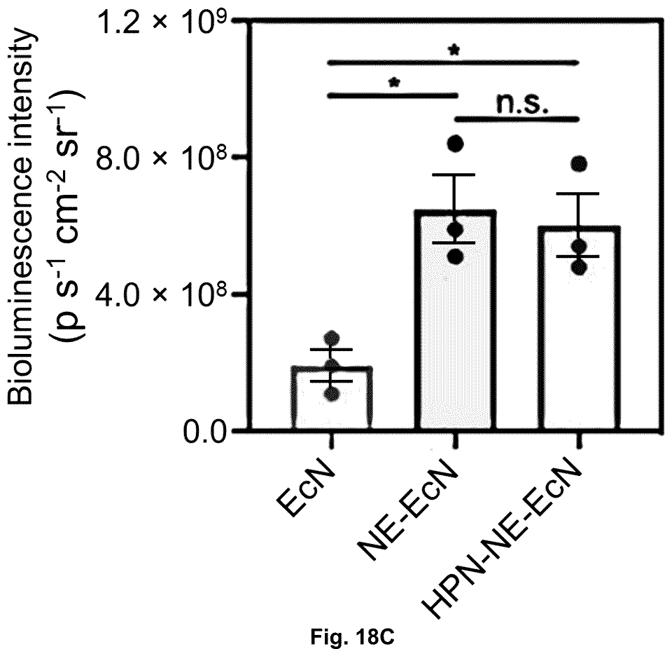

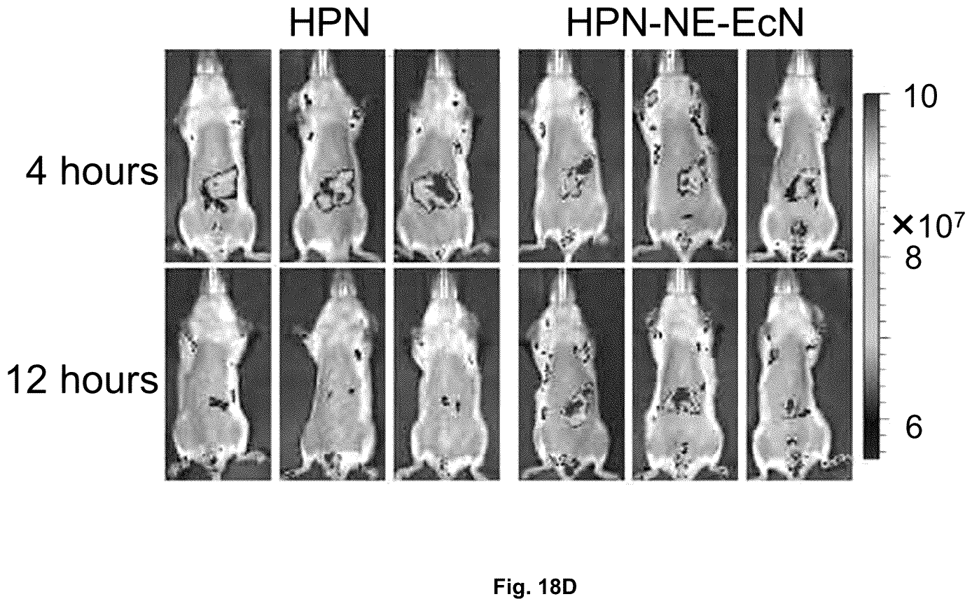

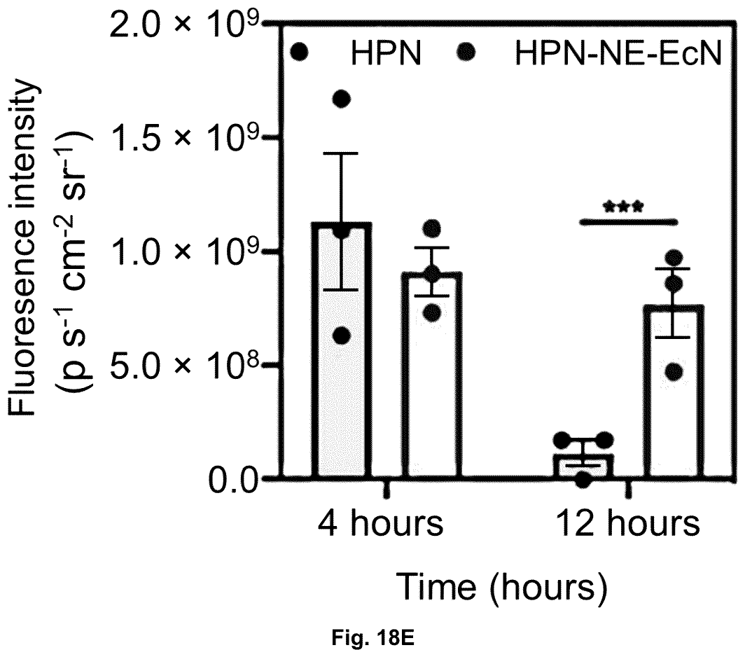

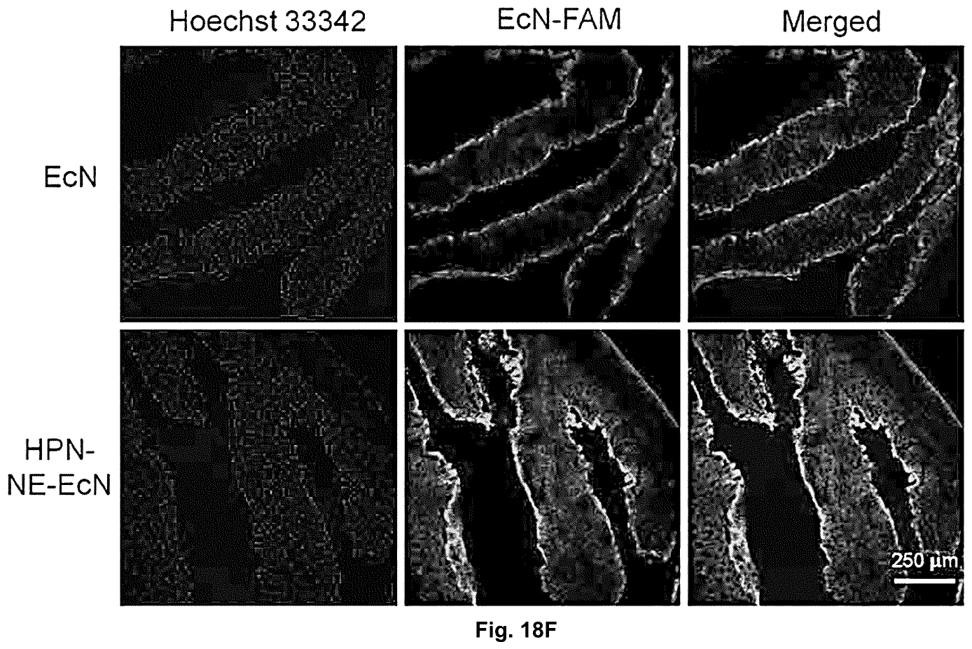

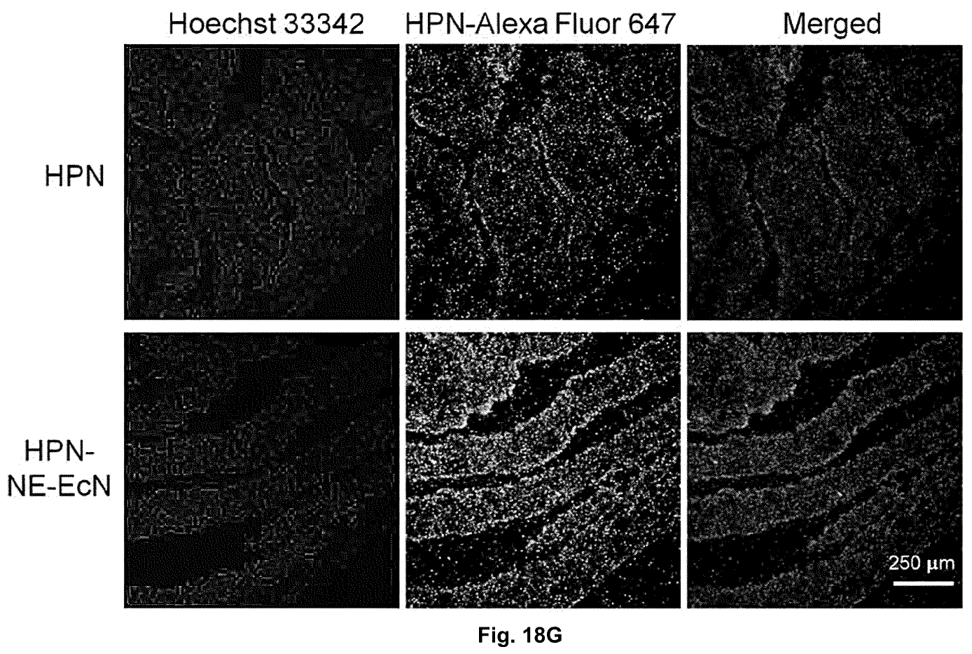

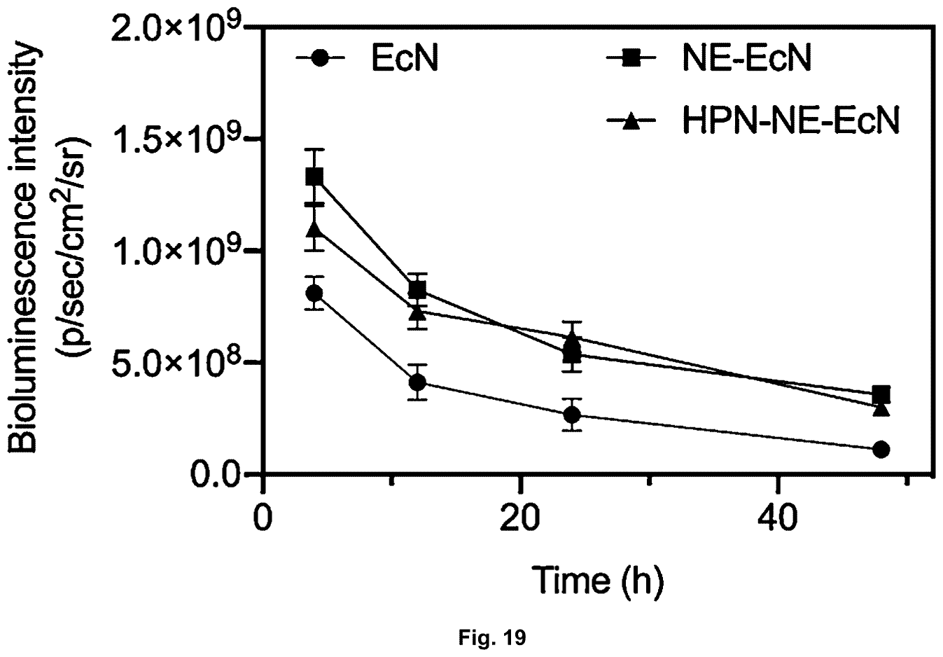

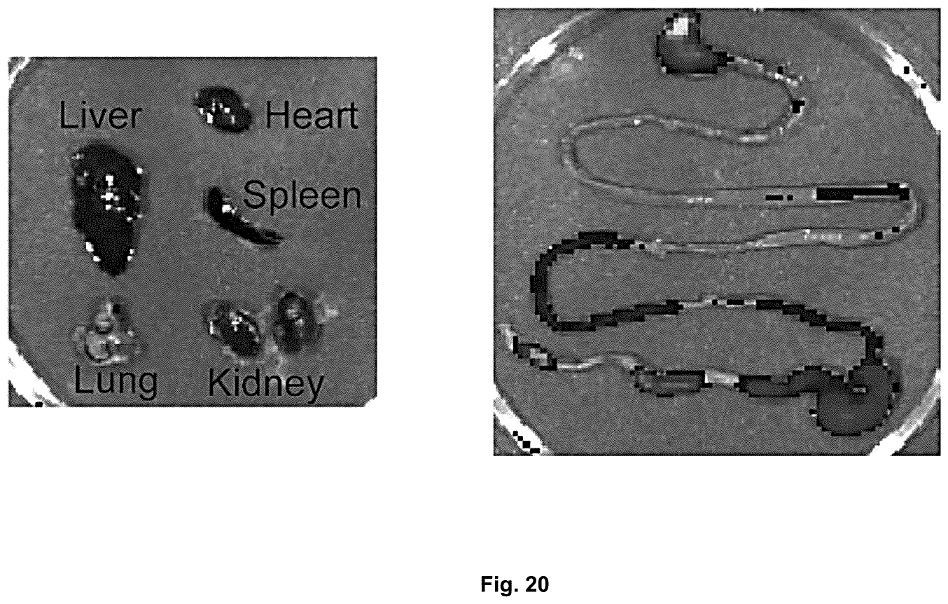

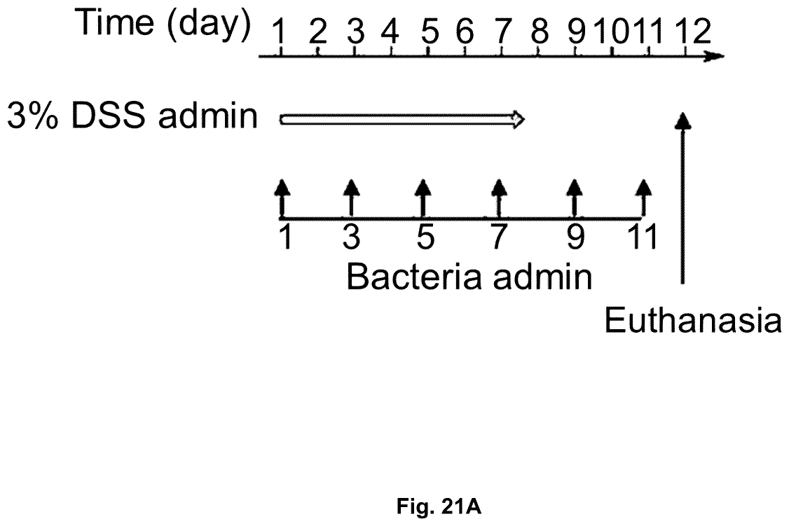

Due to the mucoadhesive capability of the NE layer, the intestinal retention time of NE-EcN and HPN-NE-EcN were investigated in vivo. The EcN strains were electro-transformed with a fluorescent reporter plasmid, PAKgfpLux2, for in vivo bioluminescence-based imaging. As shown in FIGS. 18A and 19, the mice treated with NE-EcN and HPN-NE-EcN exhibited prolonged retention time compared to the EcN group. The bioluminescence intensity of the NE-EcN and the HPN-NE-EcN groups were significantly higher than that of the EcN group at all measured timepoints, as evidenced by a slower rate of bioluminescent signal decay in the NE-EcN and the HPN-NE-EcN groups compared to the EcN group. After 48 hours, the mice were sacrificed, and the intestines were collected for ex vivo IVIS imaging. As shown in FIGS. 18B and 18C, the bioluminescence intensity of NE-EcN and HPN-NE-EcN groups were 3.4-fold and 3.2-fold higher than that of the EcN group, respectively. This demonstrates the enhanced mucoadhesive capability of bacteria endowed by the NE layer coating. Moreover, there was no significant difference in the bioluminescence intensity between the NE-EcN and HPN-NE-EcN groups, indicating that conjugation of HPN on bacterial surfaces does not negatively affect the mucoadhesive capability of the NE layer. Next, we investigated whether the conjugation of HPN on the surface of NE-EcN and the prolonged retention time of EcN after NE layer coating would reduce the clearance rate of HPN and also extend the retention time of HPN in the intestines for persistent ROS-scavenging. The HPN was first labeled with Alexa fluor 647 for IVIS imaging. As shown in FIGS. 18D and 18E, there was no significant difference in fluorescence between the HPN and HPN-NE-EcN groups at 4 h. However, after 12 h, the fluorescence intensity in the HPN group was markedly reduced compared to the HPN-NE-EcN group. The fluorescence intensity of the HPN-NE-EcN group was 7.0-fold higher than that of HPN group at 12 h, demonstrating that the conjugation of HPN on NE-EcN prolongs the retention time of HPN in the intestines. In addition, the HPN was not distributed to any other major organs including the heart, liver, spleen, lungs, and kidneys, demonstrating the specificity of the HPN to the intestines (FIG. 20).

Next, we further evaluated the mucoadhesive capability of HPN-NE-EcN by LSCM. The EcN strains were first labeled with FAM, and HPN was stained with Alexa fluor 647. After 48 h oral administration of EcN and HPN-NE-EcN, the mice were sacrificed, and colon tissues were collected for frozen slide preparation. As shown in FIG. 18F, the green fluorescence in the HPN-NE-EcN group was significantly higher than that in the EcN group, further confirming that the mucoadhesive capability of the NE layer prolonged the retention time of EcN. Moreover, after 12 h, the fluorescence of HPN, represented by the cyan coloring, in the HPN-NE-EcN group was significantly stronger than that of HPN group (FIG. 18G), demonstrating the longer retention time of HPN in the HPN-NE-EcN group compared to the HPN group.

### Prophylactic and Therapeutic Efficacy of HPN-NE-EcN Against Colitis.

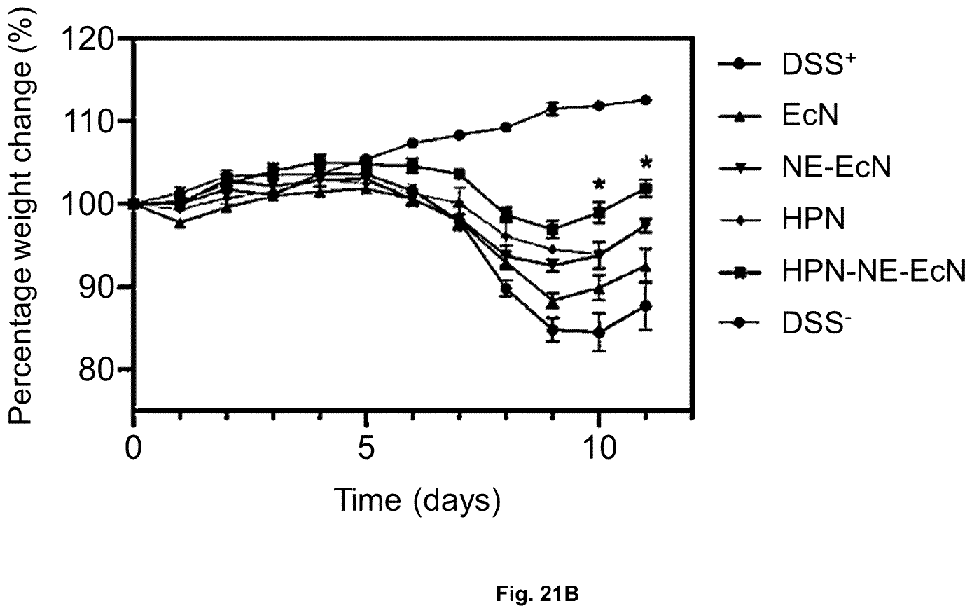

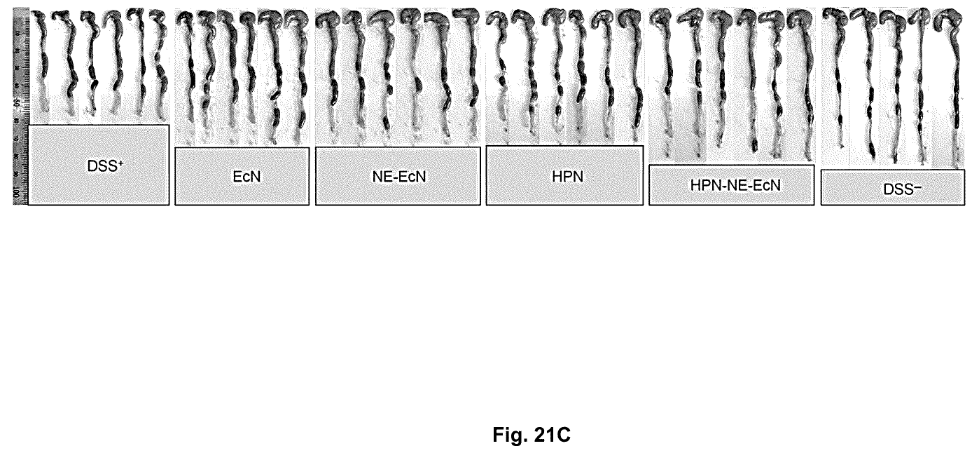

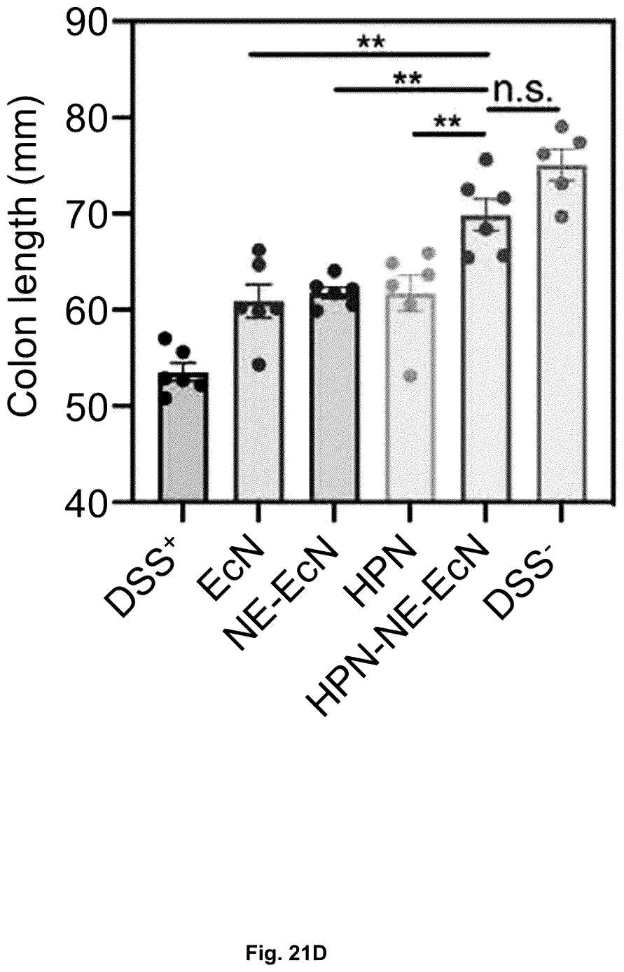

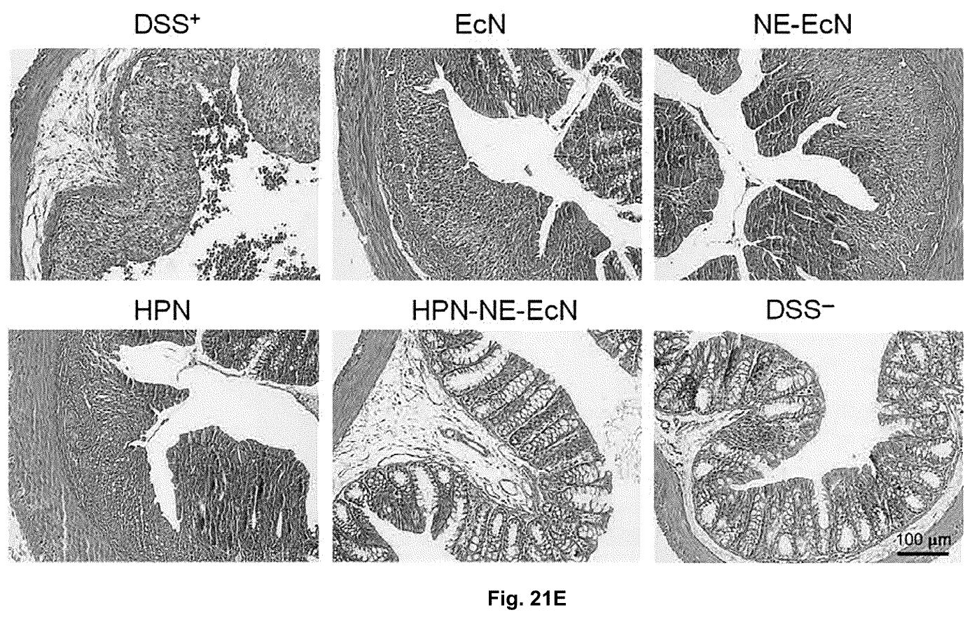

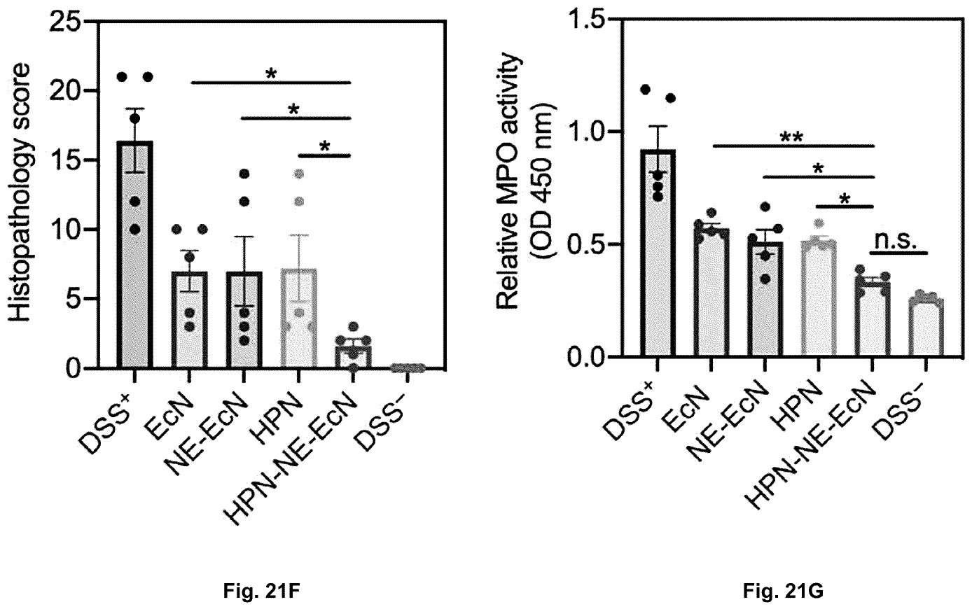

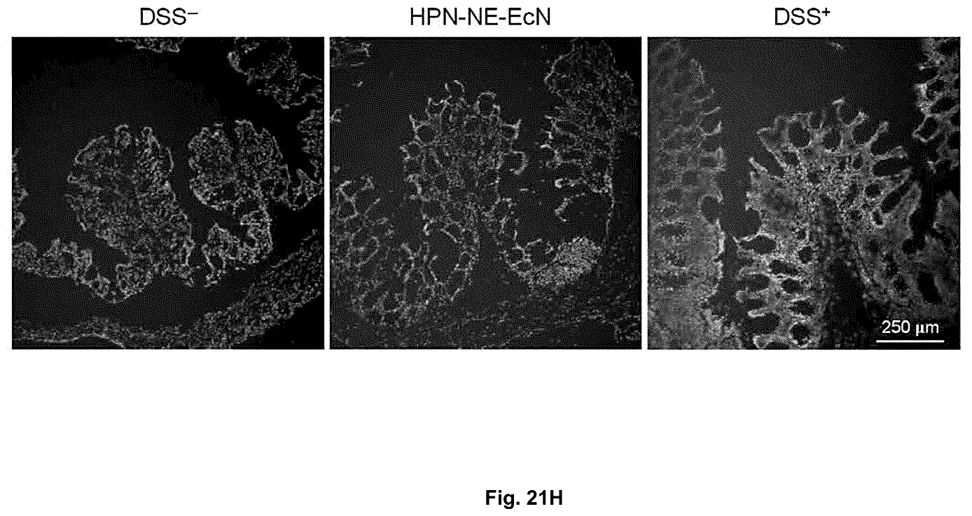

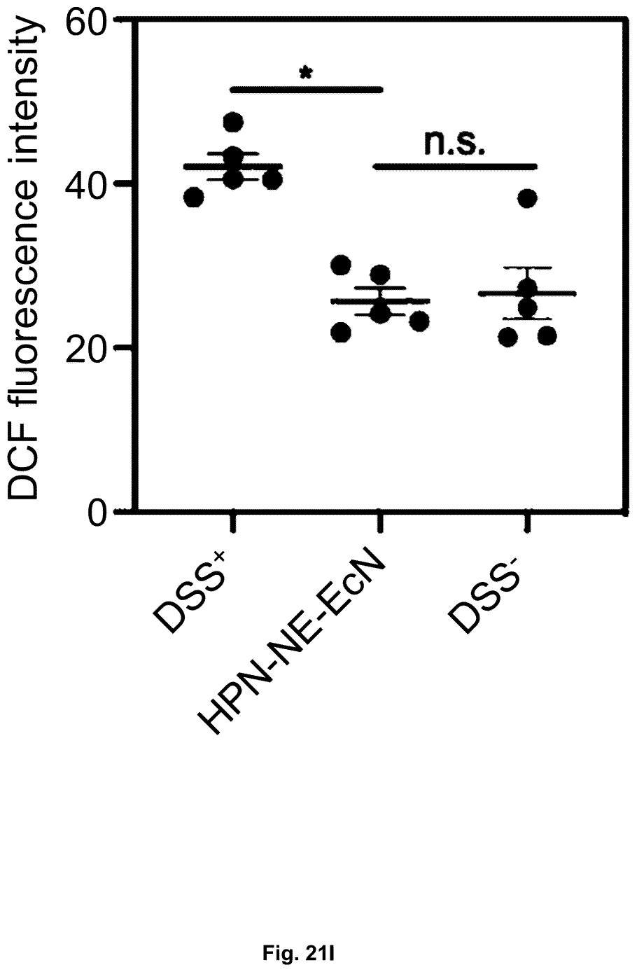

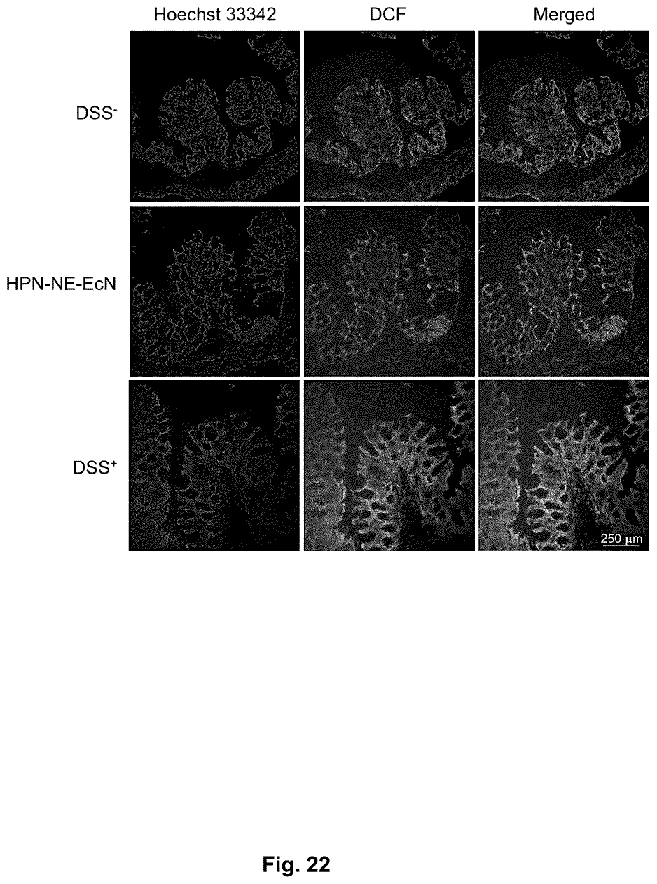

Next, the prophylactic and therapeutic efficacy of HPN-NE-EcN against colitis in vivo were evaluated. Firstly, to investigate whether HPN-NE-EcN treatment could exhibit prophylactic efficacy to protect mice from colitis development, the mice were orally administered various HPN and EcN-based formulations once every two days until they were sacrificed at day 12 (bacteria dose: 1×10⁸ CFU; HPN: 30 mg/kg). The mice were simultaneously given dextran sulfate sodium (DSS) for the first 7 days to induce colitis (FIG. 21A). The mice treated with PBS were served as the positive control and the mice without DSS treatment were set as the negative control. The changes in body weight were monitored to intuitively reflect the severity of colitis. As shown in FIG. 21B, all the DSS treatment groups displayed reduced body weight after 5 days of treatment. On day 8, all the HPN and EcN treatment groups displayed attenuated weight loss compared to the DSS⁺ group, indicating the beneficial efficacy of both HPN and EcN. Notably, the mice treated with HPN-NE-EcN showed substantially less weight loss at day 6 and had the least amount of weight loss compared to the other treatment groups. The initial body weight in the HPN-NE-EcN group was fully recovered after three days upon discontinuation of DSS therapy, demonstrating the potent prophylactic efficacy of HPN-NE-EcN against colitis.

The deleterious inflammatory response induced by colitis also causes a reduction in colon length as a result of chronic tissue damage. Therefore, mice colons were isolated and imaged (FIG. 21C), and the colon length was summarized to further evaluate colon damage. As shown in FIG. 21D, the mice treated with PBS (DSS⁺), EcN, NE-EcN, and HPN displayed an average of 28.6%, 18.8%, 17.6%, and 17.7% reduction in colon length compared to that of DSS⁻ control, respectively, while the HPN-NE-EcN group showed only an average of 6.9% reduction, further demonstrating the most prominent protective effect of HPN-NE-EcN against colitis. Next, colon damage levels were further evaluated through histological analysis to assess the treatment efficacy, and the representative histological images were presented. As shown in FIG. 21E, a complete loss of crypt, goblet cell depletion, and immune cell infiltration was observed in the DSS⁺ group, whereas substantial improvements were found in all EcN and HPN treatment groups. This was especially true for the HPN-NE-EcN group, which showed an almost intact epithelium layer and negligible inflammatory cell infiltration. Moreover, a histological scoring system (Table 1) related to inflammation severity, depth of injury, crypt cell damage, and percentage of colon tissue involvement was applied to quantitatively evaluate the colon tissue damage levels. As shown in FIG. 21F, the histological score for the HPN-NE-EcN group was significantly lower than all other DSS treatment groups, displaying a 10.2-fold, 4.4-fold, 4.4-fold, and 4.5-fold lower score than DSS⁺, EcN, NE-EcN, and HPN groups, respectively, indicating the superior efficacy of HPN-NE-EcN against colitis.

TABLE 1
Histological scoring guideline for DSS-induced colitis.

| Inflammation | | Depth of injury | | Crypt damage | | Percentage involved | |
|---|---|---|---|---|---|---|---|
| 0 | None | 0 | None | 0 | None | ×1 | 0~25% |
| 1 | Slight | 1 | Mucosa | 1 | One-third damaged | ×2 | 26%~50% |
| 2 | Moderate | 2 | Mucosa and submucosa | 2 | Two-third damaged | ×3 | 51%~75% |
| 3 | Severe | 3 | Transmural | 3 | Only surface epithelium intact | ×4 | 76%~100% |
| | | | | 4 | Entire crypt and epithelium lost | | |

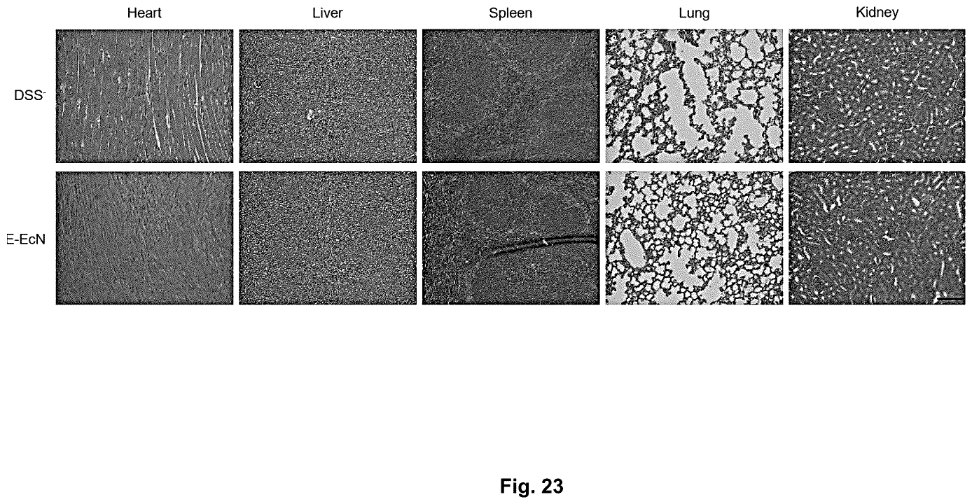

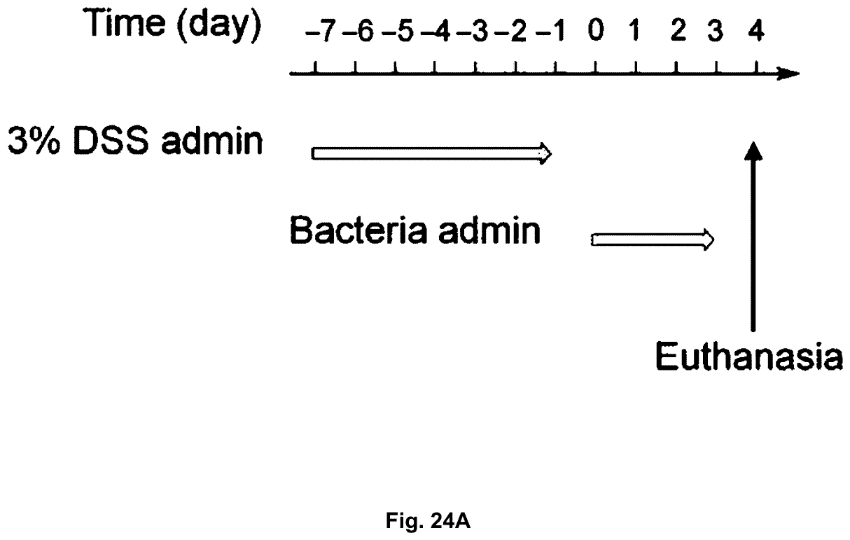

Next, colonic myeloperoxidase (MPO) activity, a marker for neutrophil activity, was measured to assess the degree of neutrophil infiltration in colon tissues. As shown in FIG. 21G, the MPO activity of the HPN-NE-EcN group was significantly lower than all other treatment groups, while showing no significant difference compared to the DSS⁻ control, indicating the potent anti-inflammatory effect from HPN-NE-EcN. To evaluate the ROS-scavenging ability of HPN-NE-EcN in vivo, the ROS levels in colon tissues were measured using DCF fluorescence imaging. The representative images were shown in FIGS. 21H and 22. It is highly apparent that the DCF fluorescence represented as green color in HPN-NE-EcN group was weaker than that of the DSS⁺ group. Meanwhile, the quantified DCF fluorescence data also revealed that the DCF intensity of the HPN-NE-EcN group was significantly lower than that of the DSS⁺ group and showed no significant difference when compared to the DSS⁻ groups (FIG. 21I). The results show recovered ROS levels in colon tissues, and indicate the substantial ROS-scavenging capability of HPN-NE-EcN. Furthermore, histological analysis of major organs (FIG. 23), including heart, liver, spleen, lung, and kidney, indicated negligible side effects caused by HPN-NE-EcN.

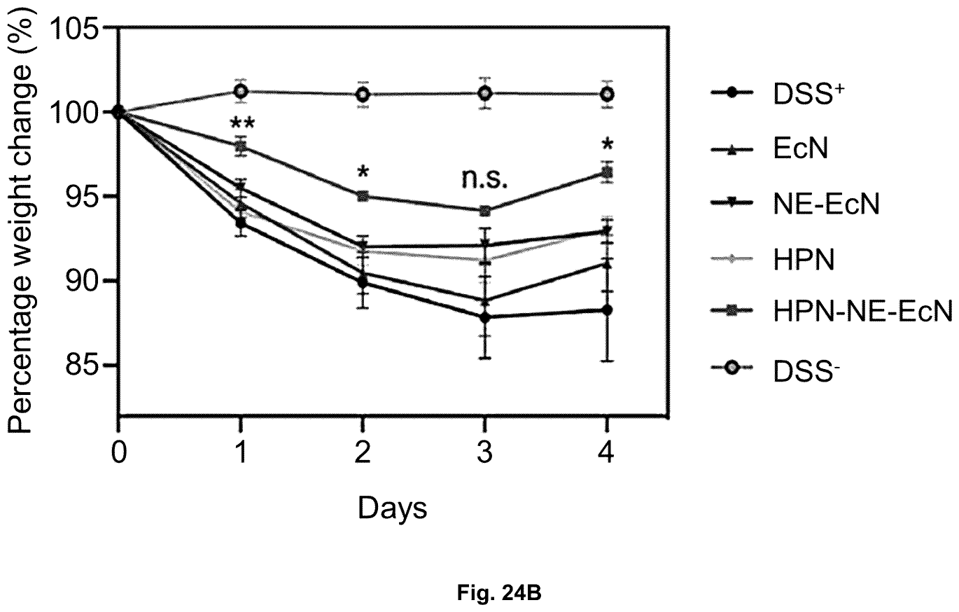

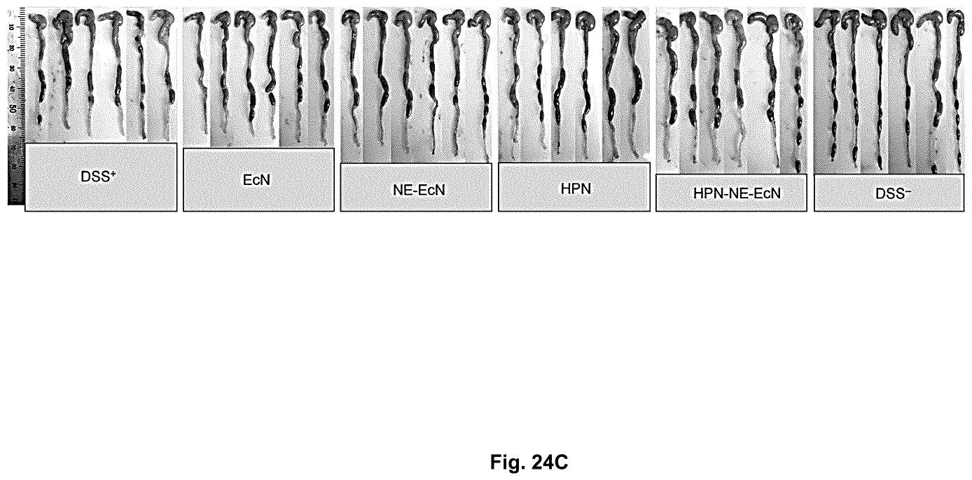

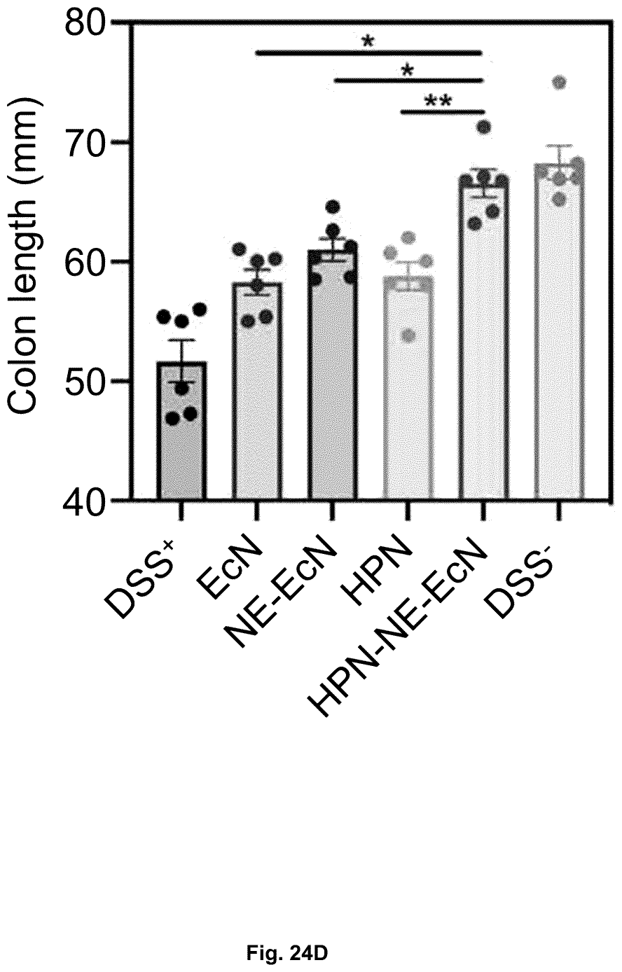

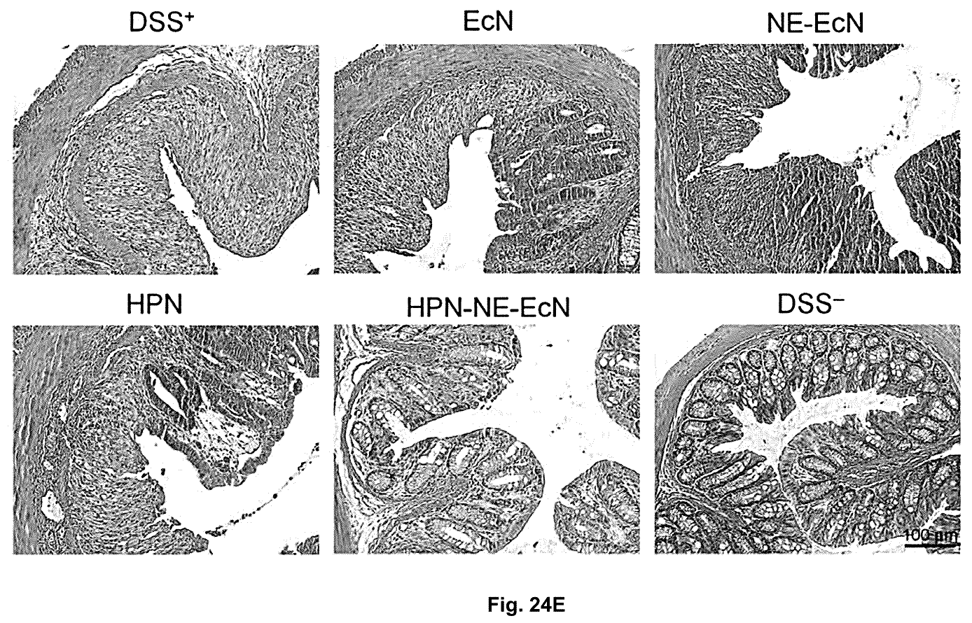

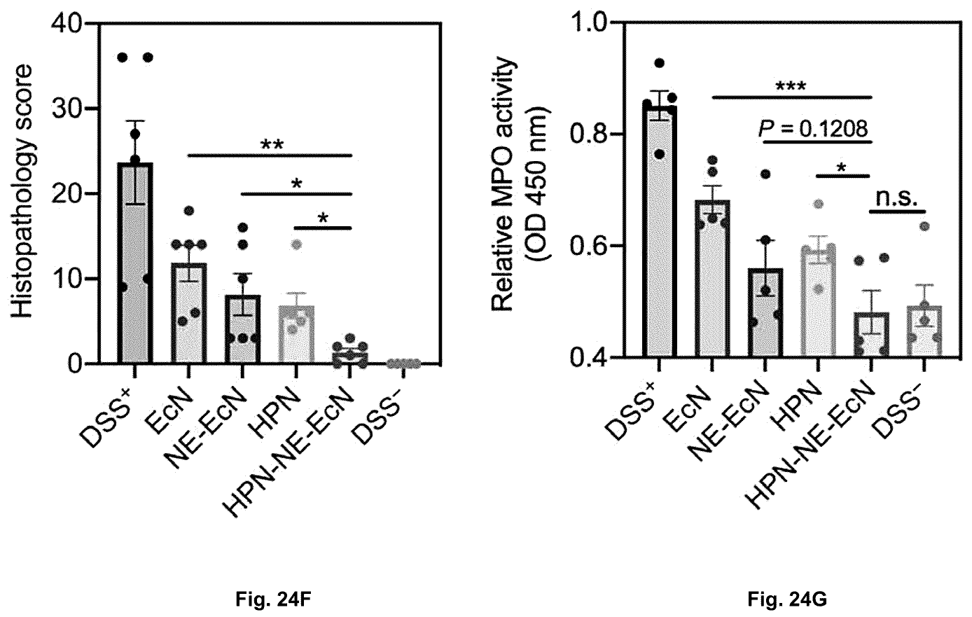

To validate the therapeutic efficacy of HPN-NE-EcN against GI tract-related diseases, the mouse colitis model was first developed by feeding DSS to mice for 7 days without treatment. Afterward, the DSS was discontinued, and different formulations of EcN and HPN (bacteria dose: 1×10⁸ CFU; HPN: 30 mg/kg) were orally administered for 4 consecutive days (FIG. 24A), and body weights were monitored. As shown in FIG. 24B, the mice of the HPN-NE-EcN group displayed ameliorated weight loss during the duration of a 4-day time-course therapy, and the weight loss was significantly lower than that of all other treatment groups from day 2. Moreover, the colon length in the HPN-NE-EcN group was significantly longer than that in the other DSS treatment groups (FIGS. 24C and 24D). Hematoxylin and eosin (H&E) staining revealed the lowest colon damage occurred in the HPN-NE-EcN group (FIG. 24E), and the histological score for the HPN-NE-EcN group was also significantly lower compared to all other DSS treatment groups (FIG. 24F). In addition, MPO activity in colon tissues of the HPN-NE-EcN group was substantially lower than that of other DSS treatment groups and had no significant difference when compared to the DSS⁻ control (FIG. 24G). Taken together, HPN-NE-EcN exhibited potent prophylactic and therapeutic efficacy for the treatment of colitis.

### CONCLUSION

Given the elevated ROS levels and disordered microbiota in the intestinal microenvironment of GI tract-related diseases, we developed a novel platform of HPN-NE-EcN capable of preventing and mitigating the detrimental effects associated with IBDs. The HPN nanoparticles, self-assembled by the synthesized polymer of HA-PPS, exhibited potent ROS-scavenging capability and could protect colonic epithelial cells, as well as the microbiome, including EcN and E. coli K12 strains from oxidative stress-induced damages. The commensal bacteria of EcN that has potential for regulating the balance of intestinal flora was further designed to be encapsulated with the NE layer on their surface. The generated NE layer provided EcN resistance against a variety of environmental assaults to improve viability during oral delivery. Moreover, the mucoadhesive capability of the NE layer endowed the EcN cells prolonged retention time in the intestines for enhanced therapeutic efficacy. Furthermore, the probiotics of EcN is an effectively colon-targeted carrier due to the natural colon tissue colonization property. Thus, except for the intestinal flora modulation property of EcN, the generated platform of HPN-NE-EcN by conjugating HPN on the surface of EcN via a ROS-responsive linker could also effectively deliver HPN to inflammatory colon tissues for targeted ROS-scavenging. Moreover, the prolonged retention time of HPN-NE-EcN due to the mucoadhesive capability of the NE layer overcame the quick clearance of HPN for persistently scavenging ROS in colon tissues. Notably, the NE layer coating strategy can be also adapted to encapsulate other live cells for cytoprotection, providing a broad application for facilitating live cell oral delivery. The synergized effectiveness of HPN-NE-EcN for simultaneously scavenging ROS and modulating microbiota balance in colonic microenvironments provided significantly enhanced prophylactic and therapeutic efficacy against DSS-induced colitis.

### REFERENCES

1. Liu, Y. et al. Integrated cascade nanozyme catalyzes in vivo ROS scavenging for antiinflammatory therapy. Science Advances 6, eabb2695 (2020).
2. Bertoni, S. et al. pH and reactive oxygen species-sequential responsive nano-in-micro composite for targeted therapy of inflammatory bowel disease. Advanced functional materials 28, 1806175 (2018).
3. Lee, Y. et al. Hyaluronic acid-bilirubin nanomedicine for targeted modulation of dysregulated intestinal barrier, microbiome and immune responses in colitis. Nat Mater 19, 118-126, doi:10.1038/s41563-019-0462-9 (2020).
4. Shi, C. et al. A nanoparticulate dual scavenger for targeted therapy of inflammatory bowel disease. Science Advances 8, eabj2372.
5. Zhong, D. et al. Orally deliverable strategy based on microalgal biomass for intestinal disease treatment. Science Advances 7, eabi9265 (2021).
6. Li, Y. et al. Acquired superoxide-scavenging ability of ceria nanoparticles. Angewandte Chemie 127, 1852-1855 (2015).
7. Wan, W.-L. et al. Photosynthesis-inspired H2 generation using a chlorophyll-loaded liposomal nanoplatform to detect and scavenge excess ROS. Nature Communications 11, 1-9 (2020).
8. Zhao, P. et al. Nanoparticle-assembled bioadhesive coacervate coating with prolonged gastrointestinal retention for inflammatory bowel disease therapy. Nature Communications 12, 1-13 (2021).
9. Chen, K. et al. A probiotic yeast-based immunotherapy against Clostridioides difficile infection. Science Translational Medicine 12, eaax4905 (2020).
10. Zhang, Y., Li, C.-X. & Zhang, X.-Z. Bacteriophage-mediated modulation of microbiota for diseases treatment. Advanced Drug Delivery Reviews 176, 113856 (2021).
11. Caruso, R., Lo, B. C. & Ndfiez, G. Host-microbiota interactions in inflammatory bowel disease. Nature Reviews Immunology 20, 411-426 (2020).
12. Li, Z. et al. Cell-based delivery systems: emerging carriers for immunotherapy. Advanced Functional Materials 31, 2100088 (2021).
13. Lin, S. et al. Mucosal immunity-mediated modulation of the gut microbiome by oral delivery of probiotics into Peyer's patches. Science Advances 7, eabf0677 (2021).
14. Pan, C. et al. Polymerization-Mediated Multifunctionalization of Living Cells for Enhanced Cell-Based Therapy. Advanced Materials 33, 2007379 (2021).
15. Liu, J. et al. Biomaterials coating for on-demand bacteria delivery: Selective release, adhesion, and detachment. Nano Today 41, 101291 (2021).
16. Li, Z. et al. Chemically and Biologically Engineered Bacteria-Based Delivery Systems for Emerging Diagnosis and Advanced Therapy. Advanced Materials 33, 2102580 (2021).
17. Anselmo, A. C., McHugh, K. J., Webster, J., Langer, R. & Jaklenec, A. Layer-by-layer encapsulation of probiotics for delivery to the microbiome. Advanced Materials 28, 9486-9490 (2016).
18. Qiu, K., Young, I., Woodburn, B. M., Huang, Y. & Anselmo, A. C. Polymeric films for the encapsulation, storage, and tunable release of therapeutic microbes. Advanced Healthcare Materials 9, 1901643 (2020).
19. Deng, H. et al. Targeted scavenging of extracellular ROS relieves suppressive immunogenic cell death. Nature Communications 11, 1-12 (2020).
20. Gupta, M. K. et al. Cell protective, ABC triblock polymer-based thermoresponsive hydrogels with ROS-triggered degradation and drug release. Journal of the American Chemical Society 136, 14896-14902 (2014).
21. Vanderburgh, J. et al. Tuning ligand density to optimize pharmacokinetics of targeted nanoparticles for dual protection against tumor-induced bone destruction. ACS Nano 14, 311-327 (2020).
22. Li, Z. et al. NIR/ROS-responsive black phosphorus QD vesicles as immunoadjuvant carrier for specific cancer photodynamic immunotherapy. Advanced Functional Materials 30, 1905758 (2020).
23. Luo, R. et al. Calcium pectinate and hyaluronic acid modified lactoferrin nanoparticles loaded rhein with dual-targeting for ulcerative colitis treatment. Carbohydrate Polymers 263, 117998 (2021).
24. Napoli, A., Valentini, M., Tirelli, N., Müller, M. & Hubbell, J. A. Oxidation-responsive polymeric vesicles. Nature Materials 3, 183-189 (2004).
25. Li, J. et al. Gastrointestinal synthetic epithelial linings. Science Translational Medicine 12, eabc0441 (2020).
26. Liu, Y., Ai, K. & Lu, L. Polydopamine and its derivative materials: synthesis and promising applications in energy, environmental, and biomedical fields. Chemical Reviews 114, 5057-5115 (2014).
27. Praveschotinunt, P. et al. Engineered E. coli Nissle 1917 for the delivery of matrixtethered therapeutic domains to the gut. Nature Communications 10, 1-14 (2019).
28. Cheng, D.-B. et al. Endogenous reactive oxygen species-triggered morphology transformation for enhanced cooperative interaction with mitochondria. Journal of the American Chemical Society 141, 7235-7239 (2019).

What is claimed is:

1. A composition of matter for treating inflammatory bowel diseases comprising a reactive oxygen species (“ROS”) scavenger conjugated to a probiotic.

2. The composition of matter of claim 1, wherein the ROS scavenger is a hyaluronic acid-poly (propylene sulfide) (“HA-PPS”) polymer that is self-assembled to form a nanoparticle (“HPN”).

3. The composition of matter of claim 2, wherein the HA-PPS polymer is formed by conjugating hyaluronic acid to poly (propylene sulfide) with ethylenediamine as a linker.

4. The composition of matter of claim 2, wherein the HPN self-degrades after reacting with a ROS.

5. The composition of matter of claim 1, wherein the probiotic is encapsulated by one or more coatings.

6. The composition of matter of claim 5, wherein the coating is formed by auto-oxidation of norepinephrine.

7. The composition of matter of claim 1, wherein the probiotic is Escherichia coli Nissle 1917.

8. The composition of matter of claim 1, wherein the ROS scavenger is conjugated to the probiotic with a ROS-responsive linker.

9. The composition of matter of claim 8, wherein the ROS-responsive linker has a terminal amine group.

10. The composition of matter of claim 8, wherein the ROS-responsive linker is 2,2′-(propane-2,2-diylbis(sulfanediyl))bis(ethan-1-amine).

11. The composition of matter of claim 8, wherein at least a portion of the ROS-responsive linker degrades in the presence of ROS to release the ROS scavenger from the probiotic.

12. A composition of matter for treating inflammatory bowel diseases comprising a reactive oxygen species (“ROS”) scavenger conjugated to a probiotic, wherein the ROS scavenger is a hyaluronic acid-poly (propylene sulfide) (“HA-PPS”) polymer that is self-assembled to form a nanoparticle (“HPN”), and wherein the HA-PPS polymer is formed by conjugating hyaluronic acid to poly (propylene sulfide) with ethylenediamine as a linker.

13. The composition of matter of claim 12, wherein the probiotic is encapsulated by one or more coatings.

14. The composition of matter of claim 13, wherein the coating is formed by auto-oxidation of norepinephrine.

15. The composition of matter of claim 12, wherein the probiotic is Escherichia coli Nissle 1917.

16. The composition of matter of claim 12, wherein the ROS scavenger is conjugated to the probiotic with a ROS-responsive linker.

17. The composition of matter of claim 16, wherein the ROS-responsive linker has a terminal amine group.

18. The composition of matter of claim 16, wherein the ROS-responsive linker is 2,2′-(propane-2,2-diylbis(sulfanediyl))bis(ethan-1-amine).

19. A pharmaceutical composition for treating IBDs, comprising the composition of matter of claim 1.

20. A method of treating IBDs, comprising administering to a subject a therapeutically effective amount of the composition of matter of claim 1.

21. The method of claim 20, wherein the composition of matter is orally administered to reach the gastrointestinal tract of a subject.

22. The method of claim 20, wherein the therapeutically effective amount of the composition of matter comprises between about 10⁶ and about 10¹² CFU probiotic and between about 1 to about 100 mg/kg ROS scavenger.
